(19) United States
(12) Patent Application Publication
(10) Pub. No.: US 2003/0224387 A1
(43) Pub. Date: Dec. 4, 2003

(54) Association of molecules with electrodes of an array of electrodes

(76) Inventors: Sandeep Kunwar, Hillsborough, CA (US); Sobha Pisharody, Castro Valley, CA (US); George Thomas Mathai, Castro Valley, CA (US); Kristian Scaboo, Castro Valley, CA (US)

Correspondence Address: PENNIE & EDMONDS LLP, 1667 K STREET NW, SUITE 1000, WASHINGTON, DC, 20006

(21) Appl. No.: 10/327,868

(22) Filed: Dec. 26, 2002

Related U.S. Application Data
(60) Provisional application No. 60/382,074, filed on May 22, 2002.

(51) Int. Cl. C12Q 1/68; G01F 1/64; G01N 27/26; G01N 33/50

(52) U.S. Cl. 435/6; 205/777.5

(57) ABSTRACT

The present invention relates to a method for selectively modifying electrodes of an array of electrodes. The array of electrodes may be used as a sensor or biosensor to determine the presence of and/or identity of each of a plurality of analyte molecules. In accordance with the present invention, electrodes of each of a number N subsets of electrodes of an array of electrodes are contacted with a respective liquid, each of which comprises a respective, different molecule. For each subset of the N subsets of electrodes, at least one of the member electrodes is deprotected to allow molecules of the respective liquid to associate with the deprotected electrode. The steps of contacting subsets of electrodes and deprotecting selected electrodes is repeated until each electrode in the array has been associated with a predetermined molecule.

### RELATED APPLICATIONS

This application claims priority, under 35 U.S.C. § 119(e), of U.S. Provisional Patent Application No. 60/382,074, filed on May 22, 2002, which application is incorporated herein, by reference, in its entirety.

### FIELD OF THE INVENTION

The present invention relates to the association of molecules with electrodes of an array of electrodes. In particular, different molecules may be selectively associated with different electrodes of an array of electrodes.

### BACKGROUND

Sensors, such as biosensors configured to determine the presence of biomolecules, are increasingly needed to rapidly perform a plurality of chemical or biochemical analyses. Exemplary biosensors may detect and/or quantify analytes using known interactions between a targeted analyte and a binding agent that is typically a biological macromolecule, such as an enzyme, receptor, nucleic acid, protein, lectin, or antibody. Preferred sensors are configured to determine the presence of and/or quantify a plurality of analytes.

When fabricating sensors having a plurality of binding agents, each binding agent may occupy a selected spatial region of the sensor, thereby allowing one binding agent to be discriminated from other binding agents. Where a plurality of binding agents is required, however, the time required to selectively spatially bind the different binding agents with the sensor becomes unduly long. Thus, the ability to rapidly and selectively associate molecules with selected surfaces while simultaneously inhibiting association of the molecules with other surfaces has importance in the fabrication of sensors.

### SUMMARY OF THE INVENTION

One aspect of the present invention relates to a method for preparing novel sensors (biosensors) that are useful for detecting a wide range of macromolecules as well as macromolecule binding events. Thus, the term “sensor” refers to a sensor that uses a molecule, which is preferably a macromolecule such as a e.g. nucleic acid, carbohydrate, protein, antibody, etc., to specifically recognize/bind to a target analyte. In some embodiments, the sensors of the present are exposed to analytes. Binding events between the molecules and the analytes are detected as measured changes in electrical signals.

In one aspect of the invention, the method relates to a method of modifying electrodes of an array of electrodes, by binding at least one respective probe molecule thereto. Prior to being modified, at least one respective, protective molecule preferably overlays each of at least two electrodes to be modified such that the at least one respective, protective molecule inhibits probe molecules from binding to the at least two electrodes. At least one respective, protective molecule may overlay each of all the electrodes to be modified.

In one embodiment, the method comprises:

(a) dissociating the at least one respective protective molecule from at least one electrode overlaid by at least one protective molecule; and

(b) contacting electrodes of each of a plurality of subsets of electrodes of the array of electrodes with a respective liquid, wherein each liquid comprises a respective, different probe molecule; and

wherein, at least one electrode is subjected to both the steps of (a) dissociating and (b) contacting and for, at least one electrode subjected to both the steps of (a) dissociating and (b) contacting, the respective, different probe molecule of the respective liquid binds to the electrode.

In some embodiments, the respective liquids may comprise at least two different liquids.

In some embodiments, at least 2 electrodes, e.g., at least 25 or at least 100 electrodes, are subjected to both the steps of (a) dissociating and (b) contacting. At least 2 electrodes, e.g., at least 25 or at least 100 electrodes, that are subjected to both the steps of (a) dissociating and (b) contacting may be members of respective, different subsets of electrodes.

In some embodiments, at least some subsets of the plurality of said subsets of electrodes comprise at least 2 member electrodes, e.g, at least 5, at least 10, or at least 20 member electrodes. In some embodiments, at least some subsets of the plurality of said subsets of electrodes comprise fewer than 100 member electrodes, e.g, fewer than 75, fewer than 50, fewer than 25, or fewer than 10 member electrodes.

In some embodiments, for at least some subsets of the plurality of said subsets of electrodes, the step of (b) contacting is performed after the step of (a) dissociating. For example, at least some electrodes may be subjected to the step of (a) dissociating while the electrodes are in contact with a first liquid, which is then removed, e.g., by rinsing, upon completion of the step of (a) dissociating. Then, the step of (b) contacting may be performed.

In some embodiments, for at least some subsets of the plurality of said subsets of electrodes, the step of (b) contacting may be performed after initiating the step of (a) dissociating. For example, the step of (b) dissociating may be begun prior to the step of contacting but not completed upon performing the step of (b) contacting so that dissociation continues during the step of (b) contacting.

In some embodiments, for at least some subsets of the plurality of said subsets of electrodes, the step of (a) dissociating may be performed while the subsets of electrodes are in contact with the respective liquids of the step of (b) contacting.

In some embodiments, the step of (b) contacting may comprise:

contacting each subset of a first portion of the plurality of said subsets with the respective liquid; and

while the subsets of the first portion of subsets remain in contact with the respective liquids, contacting each subset of a second, different portion of the plurality of said subsets with the respective liquid. For example, while performing the step of (b) contacting, at least 10, e.g., at least 25 or at least 100, of said subsets of electrodes may be in simultaneous contact with the respective liquid comprising a respective, different molecule.

In some embodiments, the step of (b) contacting may comprise simultaneously contacting at least some subsets of the plurality of said subsets of electrodes with the respective liquid.

In some embodiments, for each electrode of a plurality of the electrodes, e,g., most or all of the electrodes to be modified, the step of (a) dissociating may comprise modifying an electrical potential of the electrode, whereby the at least one respective, protective molecule dissociates from the electrode.

In some embodiments, for each electrode of a plurality of the electrodes, e,g., most or all of the electrodes to be modified, the step of (a) dissociating may comprise modifying an electrical potential difference between the electrode and a reference electrode, whereby the at least one respective, protective molecule dissociates from the electrode. For example, for each of at least 2 subsets, e.g., at least 10, at least 25, or at least 50, subsets of the plurality of said subsets of electrodes, the step of (b) contacting may further comprise contacting a reference electrode with the respective liquid, thereby electrically contacting the electrodes of the subset of electrodes and the reference electrode. For each of at least 2 subsets, e.g., at least 10, at least 25, or at least 50, subsets of the plurality of said subsets of electrodes, the step of (b) contacting may further comprise contacting a respective, different reference electrode with the respective liquid, thereby electrically contacting the electrodes of the subset of electrodes and the respective, different reference electrode. The liquid used in the step of (b) contacting preferably does not electrically connect the electrodes of the subset with the respective reference electrodes of other subsets of electrodes. For each of at least 2 subsets, e.g., at least 10, at least 25, or at least 50, subsets of the plurality of said subsets of electrodes and the respective, different reference electrode thereof, the step of (b) contacting may comprise applying at least one droplet of liquid to the subset of electrodes and reference electrode, each droplet of liquid comprising a respective, different probe molecules.

In some embodiments, for each of at least 2 subsets, e.g., at least 10, at least 25, or at least 50, subsets of the plurality of said subsets of electrodes, the step of (b) contacting may comprise applying at least one droplet of liquid to the subset of electrodes, each droplet of liquid comprising at least one of the respective, different probe molecules.

In some embodiments, the method further comprises repeating the steps of (a) dissociating and (b) contacting until a respective probe molecule is bound to each of at least 50 electrodes, e.g., at least 100, at least 500, or at least 1000 electrodes of the array. The steps of (a) dissociating and (b) contacting are preferably repeated until a respective probe molecule is bound to every electrode of the array to be modified.

In some embodiments, the probe molecules each comprise a polynucleotide. For example, probe molecules bound to different electrodes may comprise polynucleotides having different sequences from one another. The probe molecules may comprise a binding portion that binds the electrodes, the binding portion comprising sulfur.

In some embodiments, prior to performing the steps of (a) dissociating and (b) contacting, the method comprises overlaying each of a plurality of the electrodes with at least one respective, protective molecule by contacting the electrodes with a liquid comprising the at least one protective molecule, wherein at least one respective protective molecule binds to electrodes of the array. The at least one protective molecule may comprise at least one of an alkylsiloxane, an alkylthiolate, and a fatty acid. For example, the alkylthiolate may comprise an alkanethiol having from 1 to 22 carbon atoms. Examples of suitable alkanethiols include mercaptohexanol, mercaptooctanol and the like. The at least one respective, protective molecule may bind to an electrode by a sulfur group.

In some embodiments, the array of electrodes comprises a plurality of electrode pairs, wherein each electrode pair comprises first and second electrodes that are spaced apart by less than 1000 Angstroms, e.g., less than 500, less than 350, or less than 250 Angstroms. For at least one electrode pair of the plurality of said electrode pairs, the step of (a) dissociating may comprise dissociating the at least one respective, protective molecule from only the first electrode of the electrode pair. For at least one electrode pair of the plurality of said electrode pairs, the step of (b) contacting may comprise contacting both electrodes of the electrode pair with the same respective liquid comprising the same respective, different problem molecule. For at least one electrode pair of the plurality of said electrode pairs, the electrode pair is subjected to the step of (b) contacting and the first electrode only of the electrode pair is also subjected to the step of (a) dissociating, and wherein the respective, different probe molecule of the respective liquid binds only to the first electrode. For each electrode pair of at least 2 electrode pairs, e.g, at least 5, at least 25, at least 50 electrode pairs, of the plurality of said electrode pairs, the step of (a) dissociating may comprise dissociating the at least one respective, protective molecule from only the first electrode of the electrode pair. For each electrode pair of at least 2 electrode pairs, e.g, at least 5, at least 25, at least 50 electrode pairs, of the plurality of electrode pairs, the electrode pairs may belong to different subsets of the plurality of subsets of electrodes and the step of (b) contacting may comprise contacting the at least 2 electrode pairs, e.g, at least 5, at least 25, at least 50 electrode pairs, with respective liquids comprising respective, different probe molecules and for each electrode pair of at least 2 electrode pairs, e.g, at least 5, at least 25, at least 50 electrode pairs, contacted with respective liquids comprising respective, different probe molecules, only the first electrode of the electrode pair is also subjected to the step of (a) dissociating, and wherein the respective, different probe molecule of the respective liquid binds only to the first electrode. The method may further comprise, for at least one electrode pair having had the first electrode subjected to both the steps of (a) dissociating and (b) contacting: dissociating the at least one protective molecule from the second electrode of the electrode pair and contacting both electrodes of the electrode pair with a liquid comprising a probe molecule to be bound to the second electrode of the electrode pair, wherein the probe molecule to be bound to the second electrode is different from the probe molecule bound to the first electrode and wherein the probe molecule to be bound to the second electrode of electrode pair binds to the second electrode.

The probe molecule bound to one of the first and second electrodes may comprise a polynucleotide. For each electrode pair of at least 2 electrode pairs, e.g, at least 5, at least 25, at least 50 electrode pairs, the probe molecule bound to the other electrode may comprise a group that preferentially associates with double stranded polynucleotides as opposed to single stranded polynucleotides. Examples of molecular groups that preferentially associate with double stranded polynucleotides include intercalating compounds and groove binders. Upon contacting the electrode pair with a liquid comprising a target polynucleotide at least partially complementary to the first polynucleotide of the probe molecule bound the first electrode, the first and target polynucleotides will form a duplex region and an intercalating group of the molecule bound to the other electrode will intercalate with the duplex region. For each electrode pair of at least 2 electrode pairs, e.g, at least 5, at least 25, at least 50 electrode pairs, the probe molecule bound to the other electrode comprises an intercalating group and wherein, upon contacting the electrode pair with a liquid comprising a target polynucleotide at least partially complementary to the first polynucleotide of the probe molecule bound to the first electrode an electrical resistance between the first and second electrodes will be reduced.

In some embodiments, for at least one electrode to which a respective, different probe molecule is bound, the method may further comprise contacting the electrode with a liquid comprising a second protective molecule, wherein the second protective molecule also binds to the electrode.

Another aspect of the invention relates to a method of modifying electrodes of an array of electrode pairs. Each electrode pair preferably comprises a first and second electrode, wherein the first and second electrodes of the electrode pairs are to be modified by binding at least one respective probe molecule thereto. Prior to being modified, at least one respective, protective molecule preferably overlays each of the first and second electrodes of at least 1 electrode pair, e.g., at least 2, at least 10, at least 50, at least 100 electrode pairs, such that the at least one respective, protective molecule inhibits probe molecules from binding to the first and second electrodes. The method preferably comprises:

(a) dissociating the at least one protective molecule from the first electrode of at least 1 electrode pair, e.g., at least 2, at least 10, at least 50, at least 100 electrode pairs without dissociating the at least one protective molecule from the second electrode of the at least 1 electrode pair, the first and second electrodes of the at least 1 electrode pair being spaced apart by less than 1000 Angstroms, e.g., less than 500, less than 250 Angstroms; and

(b) contacting the first and second electrode of at least one electrode pair of the array of electrode pairs with a liquid comprising a first probe molecule, wherein, for at least one first electrode of at least 1 electrode pair, e.g., at least 2, at least 10, at least 50, at least 100 electrode pairs subjected to the step of (b) contacting, the first electrode is also subjected to the step of (a) dissociating, wherein the first probe molecule of the liquid binds to the first electrode.

For at least one electrode pair comprising a first electrode to which the first probe molecule was bound, the method may further comprise (c) dissociating the at least one protective molecule from the second electrode of the at least one electrode pair,(d) contacting electrodes of each of a second plurality of electrode pairs of the array of electrode pairs with a liquid comprising a second probe molecule to be bound to a second electrode of at least one electrode pair, and wherein, at least one second electrode is subjected to both the steps of (c) dissociating and (d) contacting and for, each second electrode subjected to both the steps of (c) dissociating and (d) contacting, the second probe molecule of the liquid binds to the second electrode.

The first probe molecule comprises a polynucleotide, e.g., a polynucleotide comprising a preferably terminal phosphorothiolate group. The second probe molecule may comprise an intercalating group configured to intercalate with double stranded polynucleotides.

Another aspect of the invention relates to a method of modifying electrodes of an array of electrodes, electrodes of the array to be modified by binding at least one respective probe molecule thereto. Prior to being modified, at least one respective protective molecule preferably overlays each of at least 2 electrodes, e.g., at least 5, at least 10, at least 25, at least 50 electrodes to be modified such that the at least one respective, protective molecule inhibits probe molecules from binding to electrodes of the at least 2 electrodes. The method preferably comprises (a) contacting a plurality of electrodes of the array of electrodes with a liquid comprising a probe molecule and (b) dissociating the at least one protective molecule from at least one of the electrodes in contact with the liquid comprising the probe molecule, wherein, for each electrode in contact with the liquid and subjected to the step of (b) dissociating, the probe molecule of the liquid binds to the electrode. The step of dissociating is preferably performed without first removing, e.g., without rinsing away, the liquid used in the step of (a) contacting.

In some embodiments, for at least 1 electrode, e.g., at least 2, at least 5, or at least 25 electrodes, the step of (b) dissociating comprises modifying an electrical potential of the at least 1 electrode.

In some embodiments, for at least 1 electrode, e.g., at least 2, at least 5, or at least 25 electrodes, the step of (b) dissociating comprises modifying an electrical potential difference between the at least 1 electrode and a reference electrode.

In some embodiments, the method further comprises (c) contacting a plurality of electrodes of the array of electrodes with a liquid comprising a different, probe molecule and (d) dissociating the at least one protective molecule from at least one electrode in contact with the liquid used in the step of (c) contacting, wherein, the different, probe molecule of the liquid binds to the at least one electrode. For at least one electrode, the step of (d) dissociating may comprise modifying an electrical potential of the at least one electrode, whereby the at least one molecule dissociates from the at least one electrode. For at least one electrode, the step of (d) dissociating may comprise modifying an electrical potential difference between the at least one electrode and a reference electrode, whereby the at least one molecule dissociates from the at least one electrode. The method of claim may further comprise repeating the steps of (c) dissociating and (d) contacting until a respective probe molecule is bound to each of at least 50 electrodes, e.g., at least 100 or at least 500 electrodes of the array. For example, the steps of (c) dissociating and (d) contacting may be repeated until a respective probe molecule is bound to every electrode of the array.

In some embodiments, the method further comprises, prior to performing the steps of (a) contacting and (b) dissociating, overlaying each of a plurality of the electrodes with at least one protective molecule by contacting the electrodes with a liquid comprising the at least one protective molecule, wherein at least respective one protective molecule binds to electrodes of the array. The at least one of the respective, protective molecules may comprise at least one of an alkylsiloxane, an alkylthiolate, and a fatty acid. For example, the alkylthiolate may comprise an alkane thiol having from 1 to 22 carbon atoms. For each electrode of a plurality of electrodes, the at least one respective, protective molecule may bind to the electrode by a sulfur group.

The probe molecules may comprise a polynucleotide. The polynucleotides of each of a plurality of the probe molecules may have different sequences from one another. The probe molecules may comprise a binding portion that binds the electrodes, the binding portion comprising at least one sulfur atom.

In some embodiments, the array of electrodes comprises a plurality of electrode pairs, each electrode pair comprising first and second electrodes that are spaced apart by less than 1000 Angstroms, e.g., less than 500 or less than 250 Angstroms. For at least one electrode pair of the plurality of said electrode pairs, the step of (a) dissociating may comprise dissociating the at least one respective, protective molecule from only the first electrode of the electrode pair and for at least one electrode pair of the plurality of said electrode pairs, the step of (b) contacting may comprise contacting both electrodes of the electrode pair with the same respective liquid comprising the same respective, different problem molecule. For at least one electrode pair of the plurality of said electrode pairs, the electrode pair may be subjected to the step of (b) contacting and the first electrode only of the electrode pair may also subjected to the step of (a) dissociating, the respective, different probe molecule of the respective liquid binds only to the first electrode. For each electrode pair of at least 2, e.g., at least 10, at least 50, at least 100 electrode pairs of the plurality of said electrode pairs, the step of (a) dissociating may comprise dissociating the at least one respective, protective molecule from only the first electrode of the electrode pair. For each electrode pair of at least 2, e.g., at least 10, at least 50, at least 100 electrode pairs of the plurality of electrode pairs, the electrode pairs may belong to different subsets of the plurality of subsets of electrodes and the step of (b) contacting may comprise contacting the at least two electrode pairs with respective liquids comprising a respective, different probe molecules. For each electrode pair of at least 2, e.g., at least 10, at least 50, at least 100 electrode pairs contacted with respective liquids comprising respective, different probe molecules, only the first electrode of the electrode pair may also be subjected to the step of (a) dissociating, wherein the respective, different probe molecule of the respective liquid binds only to the first electrode. For each of at least at least 1 electrode pair, e.g., at least 2, at least 10, at least 50, at least 100 electrode pairs, having had the first electrode subjected to both the steps of (b) dissociating and (c) contacting, the method further may comprise dissociating the at least one protective molecule from the second electrode of the electrode pair, contacting both electrodes of the electrode pair with a liquid comprising a probe molecule to be bound to the second electrode of the electrode pair, wherein the probe molecule to be bound to the second electrode is different from the probe molecule bound to the first electrode and wherein the probe molecule to be bound to the second electrode of electrode pair binds to the second electrode.

In some embodiments, for each electrode pair of a plurality of electrode pairs, the probe molecule bound to one of the first and second electrodes comprises a first polynucleotide. For each electrode pair of a plurality of electrode pairs, the probe molecule bound to the other electrode may comprise an intercalating group and wherein, upon contacting the electrode pair with a liquid comprising a target polynucleotide at least partially complementary to the first polynucleotide of the probe molecule bound to the first electrode, the first and target polynucleotides form a duplex region and the intercalating group intercalates with the duplex region polynucleotides.

Another aspect of the invention relates to a method of modifying electrodes of an array of electrodes, the electrodes to be modified by binding at least one respective probe molecule thereto. In some embodiments, the method comprises

(a) addressing at least one electrode of the array of electrodes with a dissociation potential;

(b) contacting electrodes of the array of electrodes with a liquid comprising a probe molecule;

(c) contacting electrodes of the array of electrodes with a liquid comprising a protective molecule; and

wherein at least a first electrode subjected to the step of (a) addressing is (i) subjected to the step of (b) contacting while not concurrently being subjected to the step of (a) addressing and (ii) subjected to the step of (c) contacting while not concurrently being subjected to the step of (a) addressing, and wherein at least one probe molecule and at least one protective molecule bind to the first electrode.

The method may further comprise repeatedly:

(d) addressing at least one different electrode with a dissociation potential;

(e) contacting electrodes of the array with a liquid comprising a different probe molecule;

(f) contacting electrodes of the array with a liquid comprising a protective molecule; and

wherein at least a second electrode subjected the step of (d) addressing is (1) subjected to a step of (e) contacting while not concurrently being subjected to a step of (d) addressing and (2) subjected to a step of (f) contacting while not concurrently being subjected to a step of (d) addressing, and wherein at least one different probe molecule and at least one protective molecule bind to the second electrode.

In some embodiments, the method may comprise:

(g) addressing at least one electrode of the array of electrodes with a dissociation potential, wherein at least one electrode that was subjected to the step of (a) addressing and was (1) subjected to the step of (b) contacting while not concurrently being subjected to the step of (a) addressing and (2) subjected to the step of (c) contacting while not concurrently being subjected to the step of (a) addressing is not subjected to the step of (g) addressing;

(h) contacting electrodes of the array of electrodes with a liquid comprising a different probe molecule;

(i) contacting electrodes of the array of electrodes with a liquid comprising a protective molecule; and

wherein at least a second electrode subjected to the step of (g) addressing is (1) subjected to the step of (h) contacting while not concurrently being subjected to the step of (g) addressing and (2) subjected to the step of (i) contacting while not concurrently being subjected to the step of (g) addressing, and wherein at least one probe molecule and at least one protective molecule bind to the second electrode.

In some embodiments, the step of (a) addressing may comprise modifying an electrical potential of the at least one electrode.

In some embodiments, the step of (a) addressing may comprise modifying an electrical potential difference between the at least one electrode and a reference electrode.

In some embodiments, the step of (c) contacting may be performed after the step of (b) contacting.

In some embodiments, the steps of (b) contacting and (c) contacting are performed after the step of (a) addressing.

In some embodiments, the method further comprises, prior to the steps of (a) addressing, (b) contacting, and (c) contacting, overlaying a plurality of the electrodes with at least one respective, protective molecule by contacting the electrodes with a liquid comprising the at least one respective, protective molecule, wherein at least one respective, protective molecule binds to electrodes of the array. The step of (a) addressing preferably dissociates the at least one protective molecule from the at least one electrode. The at least one protective molecule may comprise at least one of an alkylsiloxane, an alkylthiolate, and a fatty acid. For example, the protective molecule may comprise an alkane thiol having from 1 to 22 carbon atoms. For each electrode of a plurality of electrodes, the at least one protective molecule may bind to the electrode by a sulfur group.

The probe molecules may each comprise a polynucleotide. The polynucleotides of different probe molecules may have different sequences from one another.

The probe molecules may comprise a binding portion that binds the electrodes, the binding portion comprising sulfur.

Another aspect of the invention relates to a method of forming an electrical connection between a first electrode and a second electrode of an electrode pair. The method may comprise binding a first molecule to the first electrode, the first molecule comprising a first single stranded polynucleotide, binding a second molecule to the second electrode, the second molecule comprising an intercalating group configured to intercalate with double stranded polynucleotides, and contacting the electrode pair with a second single stranded polynucleotide at least partially complementary to the first polynucleotide, wherein the first and second polynucleotides form a duplex region and the intercalating group intercalates with the duplex region thereby forming the electrical connection between the first and second electrodes.

Binding the first molecule to the first electrode may comprise binding a sulfur group of the first molecule to the first electrode. The sulfur group may comprise a phosphorothioate group, e.g., a terminal phosphorothioate group.

In some embodiments, the second molecule may comprise a conductive oligomer disposed intermediate the intercalating group and a second portion of the second molecule that is associated with the second electrode. The second molecule may be free of polynucleotides.

Binding the second molecule to the second electrode may comprise binding a sulfur group of the second molecule to the second electrode.

The intercalating group may comprises at least one of (i) ethidium bromide or acridine and (ii) a derivative of ethidium bromide or a derivative or acridine.

In some embodiments, the method further comprises, prior to the step of binding the first molecule to the first electrode, overlaying at least one protective molecule upon the first electrode, whereby the at least one protective molecule inhibits association of the first and second molecules with the first electrode. The step of binding the first molecule to the first electrode comprises contacting the first and second electrodes with a liquid comprising the first molecule and modifying an electrical potential difference between the first electrode and a reference electrode to thereby deprotect the first electrode, whereupon the first molecule binds to the first electrode. The method may comprise, prior to the step of binding the second molecule to the second electrode, overlaying at least one protective molecule upon the second electrode, whereby the at least one protective molecule inhibits association of the first and second molecules with the second electrode; the step of binding the second molecule to the second electrode preferably comprises contacting the first and second electrodes with a liquid comprising the first molecule and modifying an electrical potential difference between the second electrode and a reference electrode to thereby deprotect the second electrode, whereupon the second molecule binds to the second electrode.

In some embodiments, the method further comprises forming a respective electrical connection between a first and a second electrode of each of a plurality of electrode pairs. For each electrode the method preferably comprises binding a first molecule to the first electrode, the first molecule comprising a first polynucleotide, binding a second molecule to the second electrode, the second molecule comprising an intercalating group configured to intercalate with double stranded polynucleotide compounds, and contacting the first and second molecules with a second polynucleotide at least partially complementary to the first polynucleotide; wherein the first and second polynucleotides form a duplex region and the intercalating group intercalates with the duplex region thereby forming the electrical connection between the first and second electrodes. The method of claim may comprise binding first molecules comprising respective, different first polynucleotides with the first electrodes of respective, different electrode pairs, whereby the first polynucleotides bound to different first electrodes will selectively form duplex regions with different, second polynucleotides.

In some embodiments, for each electrode pair, the method may comprise, prior to the step of binding the first molecule to the first electrode, overlaying at least one protective molecule upon the first electrode, whereby the at least one protective molecule inhibits binding of the first and second molecules with the first electrode. The step of binding the first molecule to the first electrode may comprise contacting the first and second electrodes with a liquid comprising the first molecule and modifying an electrical potential difference between the first electrode and a reference electrode to thereby deprotect the first electrode whereupon the first molecule binds to the first electrode. For each electrode pair, the method may comprise, prior to the step of binding the second molecule to the second electrode, overlaying at least one protective molecule upon the second electrode, whereby the at least one protective molecule inhibits binding of the first and second molecules with the second electrode, wherein the step of binding the second molecule with the second electrode comprises contacting the first and second electrodes with a liquid comprising the second molecule and modifying an electrical potential difference between the second electrode and a reference electrode to thereby deprotect the second electrode whereupon the second molecule binds to the second electrode.

In some embodiments, for each electrode pair, the step-of binding a first molecule to the first electrode may comprise contacting at least two subsets of the electrode pairs with a respective liquid, wherein each liquid comprises a respective, different first molecule and for each of at least two subsets of electrode pairs, modifying an electrical potential difference between the first electrode of at least one of the electrode pairs and a reference electrode, whereby the respective first molecule binds with the first electrode. The method may further comprise contacting at least two subsets of the electrode pairs with a respective liquid, wherein each liquid comprises a respective, different molecule and, for each of at least two subsets of electrode pairs, modifying an electrical potential difference between the first electrode of at least one of the electrode pairs and a reference electrode, whereby the respective first molecule binds to the first electrode. The steps of contacting at least two subsets of electrode pairs and modifying an electrical potential difference between the first electrode of at least one electrode pair of each subset may be repeated until each of the first electrodes has been associated with a respective first molecule.

In some embodiments, the step of associating a second molecule with the second electrode may comprise contacting a number N subsets of the electrode pairs with a respective liquid, wherein each liquid comprises a respective, different second molecule and N is an integer greater than 1 and less than the number of electrodes of the array and for each subset of the N subsets of electrode pairs, modifying an electrical potential difference between the second electrode of at least one of the electrode pairs and a reference electrode, whereby the respective second molecule binds to the second electrode. The method may further comprise contacting a number N′ subsets of the electrode pairs with a respective liquid, wherein each liquid comprises a respective, different compound and N′ is an integer greater than 1 and less than the number of electrodes of the array and, for each subset of the N′ subsets of electrode pairs, modifying an electrical potential difference between the second electrode of at least one of the electrode pairs and a reference electrode, whereby the respective second molecule binds to the second electrode.

The steps of contacting subsets of electrode pairs and modifying an electrical potential difference between the second electrode of at least one electrode pair of each subset may be repeated until each of the second electrodes has been bound with a respective second molecule.

Another aspect of the invention relates to a method of preparing a sensor. The method may comprise binding a first molecule to a first electrode, the first molecule comprising a first single stranded polynucleotide, binding a second molecule to a second electrode, the second molecule comprising an intercalating group configured to intercalate with double stranded polynucleotides, wherein, if the first electrode pair is contacted with a liquid comprising a second single stranded polynucleotide sequence at least partially complementary to the first polynucleotide sequence, the first and second polynucleotide sequences will form a duplex region and the intercalating group will intercalate with the duplex region thereby modifying an electrical characteristic of the first and second electrodes whereby the presence of the at least partially complementary polynucleotide may be determined.

Binding the first molecule with the first electrode may comprise binding a sulfur group of the first molecule with the first electrode. The sulfur group may comprise a phosphorothioate group, e.g., a terminal phosphorothioate group of a polynucleotide.

The second molecule may comprise a conductive oligomer disposed intermediate the intercalating group and a portion of the second molecule that is bound to the second electrode. The portion of the second molecule that is bound to the second electrode may comprise sulfur. The conductive oligomer may comprise at least one of a saccharide and an aromatic group. The conductive oligomer may be free of polynucleotides. The intercalating group may comprise at least one of (i) ethidium bromide or acridine and (ii) a derivative of ethidium bromide or a derivative of acridine.

The method may comprise, prior to the step of binding the first molecule to the first electrode, overlaying at least one protective molecule upon the first electrode, whereby the at least one protective molecule inhibits binding of the first and second molecules to the first electrode, wherein the step of binding the first molecule to the first electrode comprises contacting the first and second electrodes to with a liquid comprising the first molecule and modifying an electrical potential difference between the first electrode and a reference electrode to thereby deprotect the first electrode, whereupon the first molecule binds to the first electrode. Prior to the step of binding the second molecule with the second electrode, the method may comprise overlaying at least one protective molecule upon the second electrode, whereby the at least one protective molecule inhibits binding of the first and second molecules to the second electrode, wherein the step of binding the second molecule to the second electrode comprises contacting the first and second electrodes with a liquid comprising the second molecule and modifying an electrical potential difference between the second electrode and a reference electrode to thereby deprotect the second electrode whereupon the second molecule binds with the first electrode.

In some embodiments, the substrate comprises an electrode pair array comprising a number N^{a} electrode pairs, each electrode pair comprising a first and second electrode. For each electrode pair, the method may comprise binding a first molecule to the first electrode, the first molecule comprising a first polynucleotide, binding a second molecule to a second electrode, the second molecule comprising an intercalating group configured to intercalate with double stranded polynucleotide compounds. If the first electrode pair is contacted with a liquid comprising a second polynucleotide sequence at least partially complementary to the first polynucleotide sequence, the first and second polynucleotide sequences will form a duplex region and the intercalating group will intercalate with the duplex region of the first and complementary polynucleotides thereby modifying an electrical characteristic of the first and second electrodes whereby the presence of the at least partially complementary polynucleotide may be determined. The method may comprise binding first molecules comprising respective, different first polynucleotides to the first electrodes of respective, different electrode pairs, whereby the first polynucleotides bound to different first electrodes will selectively form duplex regions with different second polynucleotides.

In some embodiments, for each electrode pair, the method may comprise, prior to the step of binding the first molecule to the first electrode, binding at least one protective compound to the first electrode, whereby the at least one protective compound inhibits binding of the first and second molecules to the first electrode. The step of binding the first molecule to the first electrode may comprise contacting the first and second electrodes with a liquid comprising the first molecule and modifying an electrical potential difference between the first electrode and a reference electrode to thereby deprotect the first electrode whereupon the first molecule associates with the first electrode.

In some embodiments, for each electrode pair, the method may comprise, prior to the step of binding the second molecule to the second electrode, binding at least one protective compound with the second electrode, whereby the at least one protective compound inhibits binding of the first and second molecules to the second electrode. The step of binding the second molecule to the second electrode may comprise contacting the first and second electrodes with a liquid comprising the second molecule and modifying an electrical potential difference between the second electrode and a reference electrode to thereby deprotect the second electrode whereupon the second molecule associates with the first electrode.

In some embodiments, for each electrode pair, the step of binding a first molecule with the first electrode may comprise contacting a number N subsets of the electrode pairs with a respective liquid, wherein each liquid comprises a respective, different first molecule and N is an integer greater than 1 and less than N^{a} and, for each subset of the N subsets of electrode pairs, modifying an electrical potential between the first electrode of at least one of the electrode pairs and a reference electrode, whereby the respective first molecule binds to the first electrode. The method may further comprise contacting a number N′ subsets of the electrode pairs with a respective liquid, wherein each liquid comprises a respective, different compound and N′ is an integer greater than 1 and less than N′ and, for each subset of the N′ subsets of electrode pairs, modifying an electrical potential between the first electrode of at least one of the electrode pairs and a reference electrode, whereby the respective first molecule binds to the first electrode. The steps of contacting subsets of electrode pairs and modifying an electrical potential may be repeated until each of the first electrodes has been bound to a respective first molecule.

For each of the N subsets of electrode pairs, contacting the subset with a respective liquid may comprise applying at least one aliquot of the respective liquid to the subset. The electrode pairs of each subset of electrode pairs may be isolated from aliquots of liquid applied to other subsets of electrode pairs.

Another aspect of the invention relates to a method of forming an electrical connection between a first electrode and a second electrode of an electrode pair, the electrode pair comprising the first and second electrodes, wherein a surface of the first electrode is bound with a first molecule, the first molecule comprising a first single stranded polynucleotide and a surface of the second electrode is bound with a second molecule, the second molecule comprising an intercalating group configured to intercalate with double stranded polynucleotides. The method may comprise contacting the first and second molecules with a second single stranded polynucleotide at least partially complementary to the first polynucleotide, wherein the first and second polynucleotides form a duplex region and the intercalating group intercalates with the first and second polynucleotides thereby forming the electrical connection between the first and second electrodes. An electrical characteristic, e.g., a conductance, a resistance, an impedance, or a capacitance, of the first and second electrodes may be modified whereby the presence of the second polynucleotide may be determined.

Another aspect of the invention relates to an apparatus for preparing an array of modified surfaces. The apparatus may comprise a device configured to at least contact electrodes of each of a number N subsets of electrodes an array of electrodes with a respective liquid, wherein each liquid comprises a respective, different compound and N is an integer greater than 1 and, for each subset of the N subsets of electrodes, modify an electrical potential between at least a first electrode of the subset of electrodes and a reference electrode, whereby the respective compound of the fluid contacting the first electrode associates with the first electrode.

The device may be configured to at least contact surfaces of each of a number N′ subsets of the electrodes of the array of electrodes with a respective liquid, wherein each liquid comprises a respective, different compound and N′ is an integer greater than 1 and, for each subset of the N′ subsets of electrodes, modify an electrical potential between at least a second electrode and a reference electrode, whereby the respective compound associates with the second electrode.

In some embodiments, the device may be configured to repeatedly contact subsets of surfaces of the array of surfaces with a respective liquid, each liquid comprising a respective, different compound and modify an electrical potential between at least one electrode of the subset of electrodes and a reference electrode until a respective, different compound has been associated with each electrode of the array of electrodes.

The device may comprise one or more droplet preparation devices, wherein each droplet preparation device is in fluid communication with a respective reservoir comprising a respective one of the different compounds and a droplet delivery device configured to deliver droplets prepared by the one or more droplet preparation devices to predetermined subsets of the N subsets of electrodes to thereby contact the predetermined subsets with respective liquid. The droplet preparation devices may each comprise a capillary configured to prepare a droplet of fluid. The droplet preparation devices may be configured to prepare droplets by at least one of thermally modifying a pressure of the liquid, piezo-electrically modifying a pressure of the liquid, and ultrasonically modifying a pressure of the liquid.

In some embodiments, the device is configured to bind at least one protective compound to the electrodes of the array, whereby the at least one protective compound inhibits association of the respective, different compounds with surfaces.

Another aspect of the invention relates to a sensor, comprising a substrate comprising a first electrode pair comprising first and second electrodes, a first molecule bound with the first electrode, the first molecule comprising a first polynucleotide, a second molecule bound with the second electrode, the second molecule comprising a group configured to intercalate with double stranded polynucleotide compounds and wherein, upon contacting the first electrode pair with a liquid comprising a second polynucleotide sequence at least partially complementary to the first polynucleotide sequence, the first and second polynucleotide sequences form a duplex region and the intercalating portion intercalates with the at least partially annealed polynucleotides thereby modifying an electrical characteristic of the first and second electrodes whereby the presence of the at least partially second polynucleotide may be determined.

The substrate may comprise a number N^{a} electrode pairs, with each electrode pair comprising a first and second electrode pair. Each electrode pair may comprise a first molecule bound with the first electrode, the first molecule comprising a first polynucleotide, a second molecule bound with the second electrode, the second molecule comprising a group configured to intercalate with double stranded polynucleotide compounds and wherein, upon contacting the electrode pair with a liquid comprising a second polynucleotide sequence at least partially complementary to the first polynucleotide sequence, the first and second polynucleotide sequences may form a duplex region and the intercalating portion intercalates with the duplex region thereby modifying an electrical characteristic of the first and second electrodes whereby the presence of the at least partially second polynucleotide may be determined.

Respective, different first polynucleotides may be bound with the first electrodes of respective, different electrode pairs, whereby the first polynucleotides associated with different first electrodes will selectively form duplex regions with different second polynucleotides. A distance between the first and second electrodes may be less than 500 Angstroms.

### BRIEF DESCRIPTION OF THE DRAWINGS

The present invention is described below in reference to the Drawings in which:

FIG. 1 shows a top view of an exemplary biosensor in accordance with the present invention;

FIG. 2 shows a partial cross-sectional side view of a first embodiment of the biosensor of FIG. 1, the cross-section taken along a section 2;

FIG. 3 shows a partial cross-sectional side view of a second embodiment of the biosensor of FIG. 1, the cross-section taken along a section 2;

FIG. 4 shows a flow chart of exemplary steps for preparing an array of surface modified electrodes in accordance with the present invention; and

FIG. 5a shows electrodes of an array of electrodes in accordance with the present invention, the electrodes being in contact with a liquid comprising a protective molecule;

FIG. 5b shows the array of FIG. 5a, electrodes of the array each comprising a protective layer;

FIG. 5c shows the array of FIG. 1, subsets of electrodes of the array being in contact with respective liquids;

FIG. 5d shows the array of FIG. 1, an electrode of respective subsets of electrodes having been associated with a different molecule;

FIG. 5e shows the array of FIG. 1, subsets of electrodes of the array being in contact with respective liquids;

FIG. 5f shows the array of FIG. 1, two electrodes of respective subsets of electrodes having been associated with a different molecule;

FIG. 6a shows a subset of electrodes of an array of electrodes in accordance with the present invention, the subset of electrodes being in contact with a liquid comprising a probe molecule, other electrodes of the array not being shown;

FIG. 6b shows the subset of electrodes of FIG. 6a, the first probe molecule having bound to electrodes of the subset;

FIG. 6c shows the subset of electrodes of FIG. 6b, the electrodes being in contact with a protective molecule;

FIG. 6d shows the subset of electrodes of FIG. 6c, the probe molecule of FIG. 6a and the protective molecule of FIG. 6c being bound to electrodes of the subset;

FIG. 6e shows the subset of electrodes of FIG. 6d, the electrodes being in contact with a liquid comprising a different probe molecule, one of the electrodes having been addressed, with a dissociation potential;

FIG. 6f shows the subset of electrodes of FIG. 6e, the different probe molecule being bound to the electrode addressed with a dissociation potential, the electrodes of the subset being in contact with a liquid comprising a protective molecule;

FIG. 6g shows the subset of electrodes of FIG. 6f, the different probe molecule and the protective molecule being bound to an electrode of the subset;

FIG. 6h shows the subset of electrodes of FIG. 6g, the subset of electrodes having been contacted with liquids comprising two additional probe molecules;

FIG. 7a shows a subset of electrodes of an array of electrodes in accordance with the present invention, the subset of electrodes being in contact with a liquid comprising a protective molecule;

FIG. 7b shows the subset of electrodes of FIG. 7a, probe molecules being bound to electrodes of the subset;

FIG. 7c shows the subset of electrodes of FIG. 7b, the electrodes being in contact with a liquid comprising a probe molecule, one of the electrodes of the array having been addressed with a dissociation potential;

FIG. 7d shows the subset of electrodes of FIG. 7c, probe molecules being bound to one of the electrodes;

FIG. 7e shows the subset of electrodes of FIG. 7e, the electrodes being in contact with a liquid comprising a protective molecule;

FIG. 7f shows the subset of electrodes of FIG. 7e, probe molecules and protective molecules being bound to one of the electrodes of the subset;

FIG. 7g shows the subset of electrodes of FIG. 7f, the electrodes being in contact with a liquid comprising a different probe molecule;

FIG. 7h shows the subset of electrodes of FIG. 7g, the different probe molecule being bound to one of the electrodes of the subset;

FIG. 7i shows the subset of electrodes of FIG. 7h, the electrodes being in contact with a liquid comprising a protective molecule;

FIG. 7j shows the subset of electrodes of FIG. 7i, different probe molecules and protective molecules being bound to an electrode of the array;

FIG. 7k shows the subset of electrodes of FIG. 7j, the electrodes having been contacted with liquids comprising two additional probe molecules;

FIG. 8a shows the biosensor of FIG. 2, electrodes of the biosensor having a protective layer associated therewith;

FIG. 8b shows the biosensor of FIG. 6a, two of the electrodes having been associated with a probe molecule comprising a polynucleotide;

FIG. 8c shows the biosensor of FIG. 6b, two of the electrodes having been associated with a molecule having an intercalating group;

FIG. 8d shows the biosensor of FIG. 6c, the electrodes having been contacted with polynucleotides at least partially complementary to the respective polynucleotides of the probe molecules;

FIG. 8e shows the biosensor of FIG. 6d, the intercalating groups having formed intercalation complexes with the probe molecules and at least partially complementary polynucleotides;

FIGS. 9a and 9b show molecules comprising a polynucleotide comprising at least one terminal phosphorothiate group in accordance with the present invention;

FIG. 10 shows an exemplary embodiment of an apparatus configured to prepare arrays of surface modified electrodes in accordance with the present invention and

FIG. 11 shows the array of FIG. 1, liquid contacting a plurality of subsets of electrodes of the array.

### DETAILED DESCRIPTION OF PREFERRED EMBODIMENTS

The present invention relates to the selective association of molecules, such as oligonucleotide probes, with surfaces of a sensor. The surfaces may be the electrodes that are configured to determine when a probe molecule associated with the electrode has hybridized with a target nucleotide containing compound, such as a single stranded polynucleotide. Such sensors may comprise a plurality of electrodes with different nucleotide sequences associated with different electrodes. The different nucleotide sequences hybridize with different target nucleotide containing compounds thereby allowing rapid determination of the presence of a plurality of such compounds. To allow determination of a plurality of different target nucleotide containing compounds, however, sensors require numerous electrodes. The high packing density of the electrodes may complicate the preparation of the sensors. For example, conventional liquid dispensing technologies lack the resolution to dispense a liquid comprising a particular probe to be associated with only single electrode of an array of electrodes.

The present invention provides a method for selectively associating different molecules, such as different polynucleotides, with different electrodes of an array of electronically addressable electrodes.

The present invention may be used to associate molecules with various surfaces of biosensors having different surface configurations. Suitable biosensor configurations comprise those disclosed in related application No. to be assigned, filed Dec. 26, 2002, titled “DEVICE STRUCTURE FOR CLOSELY SPACED ELECTRODES,” invented by Kunwar et al. and having attorney docket number 11210-018999 and incorporated herein by reference in its entirety. Each biosensor configuration provides unique advantages. For example, some biosensor configurations are advantageous because of their ease of manufacture. Other biosensor configurations of the present invention are advantageous because of the electrical isolation they provide between electrodes within the biosensor. This electrical isolation lowers leakage currents. Still other biosensors of the present invention are advantageous because of their enhanced assay sensitivity.

Illustrative Biosensor

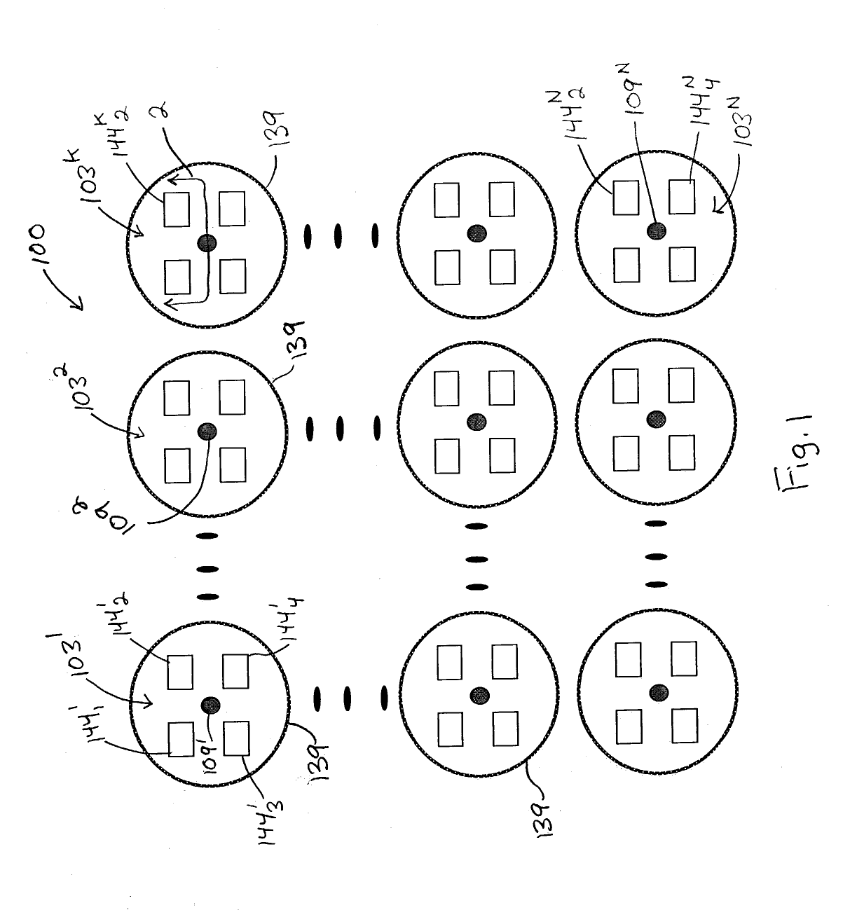

FIG. 1 illustrates a top view of a novel biosensor 100 in accordance with one embodiment of the present invention. Biosensor 100 comprises a number N^{a} sensing devices 144, where the number N^{a} is an integer, preferably at least 2, such as at least 100, e.g., at least 1000, or even 10,000 or more. Sensing devices may be supported by a substrate 102, such as a silicon wafer. It will be appreciated that each device 144 may serve as an independent sensor for a particular application. For example, each sensing device 144 may be configured to determine the presence of a different molecule, such as a polynucleotide. Sensing devices 144 may be grouped in a number N subsets of sensing devices, where the sensing devices within each subset have an index i, where i=1, 2, 3, . . . S, and the subsets have an index k, where k=1, 2, 3, . . . N^{a}. Thus, the ith sensing device of the kth subset of sensing devices may be designated as 144ᵢ^{k}. In the embodiment shown in FIG. 1, each subset comprises 4 sensing devices 144. However, the number S of sensing devices in each subset may be as small as 1. Preferably, S is at least 2, for example, at least 4, such as greater than 10, or even greater than 50. For each subset, the number of sensing devices S is preferably less than 1000, such as less than 500 or 100, for example, less than 25. The number of sensing devices S within the subsets may be different for different subsets of sensing devices.

Each subset of sensing devices preferably comprises at least one associated reference surface, each of which is preferably a reference electrode 109^{k}, where k refers to the subset index. For example, reference electrode 109² is associated with the second subset of sensing devices. The reference electrodes may be any material to which an electrical potential of another material, preferably conducting material, may be referenced. Thus, the reference electrode may comprise, for example, any reference electrode generally used in electrochemistry. A preferred reference electrode is a Ag/AgCl, which may be used with or without a salt bridge.

Biosensor 100 may comprise a plurality of liquid barriers 139, which preferably have a lower surface energy than substrate 102. For example, liquid barriers 139 may comprise a plurality of hydrophobic molecules. For example, molecules having a fluorinated or chlorinated alkyl group may be bound to a silicon comprising substrate using silane chemistry. The liquid barriers 139 may be formed by, for example, photolithography.

Microcontacting printing may also be used to print hydrophobic or hydrophilic molecules onto the substrate. Suitable microcontact printing techniques are disclosed in T. Pompe et al. Submicron Contact Printing on Silicon Using Stamp Pads, Langmuir, 15, 2398-2401, 1999, which is hereby incorporated by reference. Microcontact printing may be performed, for example, using stamps prepared by, e.g., casting, from poly(dimethylsiloxane) (PDMS) or other suitable material. Stamps may be prepared from a master having a shape complementary to the stamps. Imprinting is performed using a solution of the hydrophobic molecules and a preferably organic solvent, such as a linear or branched alkane.

Referring to FIG. 2, a cross-sectional side view of the kth subset 103^{k} of sensing devices 144 is shown. Each sensing device preferably comprises at least one surface comprising a conductive, semi-conductive, or resistive material. An electrical potential or voltage associated with the surface is preferably addressable independently of electrodes of other sensing devices. Exemplary conductive materials comprise Au, Pd, Pt, Ag, Cr, Hg, Fe, Cu, Al, Ti, and alloys comprising these materials, such as Au/Pd, Au/Ag, Ag/Pd, GaAs. Other conductive materials, such as doped semiconductors and other conductive or semiconductive inorganic or organic materials, such as 7,7′,8,8′-tetracyanoquinonedimethane (TCNQ), may also be used. In embodiments in accordance with FIG. 2, each subset 144 comprises materials 106 and 110, which are preferably independently addressable electrodes comprising a conductive or semi-conductive material.

As illustrated in FIG. 2, each sensing device 144 may comprise a spacer 140 and materials 106 and 110. In instances where materials 106 and 110 are electrodes, each device 144 may have an electrode-insulator-electrode configuration. Electrodes 106ᵢ and 110ᵢ of the ith sensing device may be referred to as an electrode pair. For example, an electrode pair of device 144₁^{k} comprises a first electrode 106-1 and a second electrode 110-1. In some embodiments, electrode pairs in accordance with the invention are separated by a distance of 10,000 Angstroms or less, e.g., 5,000 Angstroms or less. For example, electrode pairs may be separated by a distance of 1,000 Angstroms or less, e.g., 500 Angstroms or less, such as 200 Angstroms or less.

In some embodiments, a predetermined distance 121 along the z-dimension separates the top of material 106 and the top of material 110. In some embodiments, materials 106 and 110 are made of conductive, semi-conductive, or resistive materials. In some embodiments, predetermined distance 121 is achieved by overlaying material 110 on a spacer 140.

An advantage of the present invention is that predetermined distance 121 can be precisely controlled by separating materials 106 and 110 in the z dimension (FIG. 2) rather than the x dimension or the y dimension (perpendicular to the plane of FIG. 1). Separation in the z dimension is controlled using precise semiconductor manufacturing techniques that are described in more detail in related application titled “DEVICE STRUCTURE FOR CLOSELY SPACED ELECTRODES” and referenced above. The ability to precisely control the separation (distance 121) of closely spaced materials 106 and 110 has use in a broad range of fields. Examples comprise, but are not limited to, the construction of biosensors, the assembly of nanocircuits and other nanostructures, computer memory, electronic and computer switches, material science, construction, surface science, medical devices, medical therapeutics and more.

In one embodiment of the present invention, materials 106 and 110 are electrodes. One or more molecules may be coupled with electrodes 106 and 110, e.g., by binding the one or more molecules to the electrode. The one or more molecules may comprise a linker or functional group through which the molecule is coupled to the electrode. Binding preferably takes place through a covalent bond between the molecule and the electrode. For example, a molecule may be coupled to a gold or a platinum electrode by a bond comprising a sulfur group of the molecule and the gold or platinum electrode. Alternatively, or in combination with a covalent bond, binding may occur through an ionic bond or other physio-chemical interaction that retains the coupling between the molecule and the surface, preferably unless it is intended to dissociate the molecule from the surface.

Molecules bound to an electrode in accordance with the invention and useful for determining the presence of a target molecule may be referred to as probe molecules. Generally speaking, probe molecules may be coupled to electrodes 106 and 110 in such a manner that a sufficient portion of the molecule is not sterically hindered so that the molecule may interact with a “cognate” target molecule. For example, the target molecule may comprise a portion that is at least partially complementary to the probe molecule. The partially complementary probe and target molecules may interact by associating or binding. For example, probe molecule comprising a single stranded polynucleotide may interact with a target molecule comprising an at least partially complementary single stranded polynucleotide by forming a double stranded polynucleotide.

When a molecule binds or otherwise associates with its cognate target molecule, a binding agent/target molecule complex is formed, which complex may reduce a resistance between electrodes 106 and 110 of a sensing device. This change in resistance is readily detected indicating the presence and/or concentration of a molecule associated with a sensing device 144 of the biosensor 100.

In reference to FIGS. 1 and 2, one embodiment of the present invention provides a biosensor 100 comprising a plurality of devices 144 on a substrate 102. Each device 144 in the plurality of devices 144 occupies a different region on an optional insulator layer 104. The optional insulator layer 144 is overlaid on substrate 102. Furthermore, each device 144 in the plurality of devices comprises (i) a first electrically conducting material 106 having a top surface, wherein the first electrically conducting material 106 is overlaid on a first portion of optional insulator layer 104, (ii) a spacer 140 overlaid on a second portion of the insulator layer 104, and (iii) a second electrically conducting material 110 overlaid on a portion of spacer 144. As illustrated in FIG. 1, the first electrically conducting material 106 and spacer 144 abut each other. Furthermore, for any given device 144 in the plurality of devices, the first portion of insulator layer 104 occupied by the device does not overlap with the second portion of insulator layer 104 occupied by the device. As used herein, a device 144 “occupies” that portion of insulator layer 104 which is overlaid by a component (e.g., material 106, spacer 140, etc.) of the device. In embodiments where insulator 104 is not used, each device 144 occupies a portion of substrate 102 and material 106 and spacer 140 each directly overlay a portion of substrate 102.

In some embodiments in accordance with FIG. 2, a distance between a plane comprising the top surface of the first electrically conducting material 106 and a plane comprising the top surface of the second electrically conducting material 110 is less than 500 Angstroms. In some embodiments of the present invention, the distance between a plane comprising the top surface of the first electrically conducting material 106 and a plane comprising the top surface of the second electrically conducting material 110 is less than 250 Angstroms. In still other embodiments, a distance between a plane comprising the top surface of the first electrically conducting material and a plane comprising the top surface of the second electrically conducting material is less than 100 Angstroms. In still other embodiments of the present invention, a distance between a plane comprising the top surface of the first electrically conducting material 106 and a plane comprising the top surface of the second electrically conducting material 110 is between about 40 Angstroms and about 60 Angstroms.

Illustrative Biosensor with Overlapping Electrodes

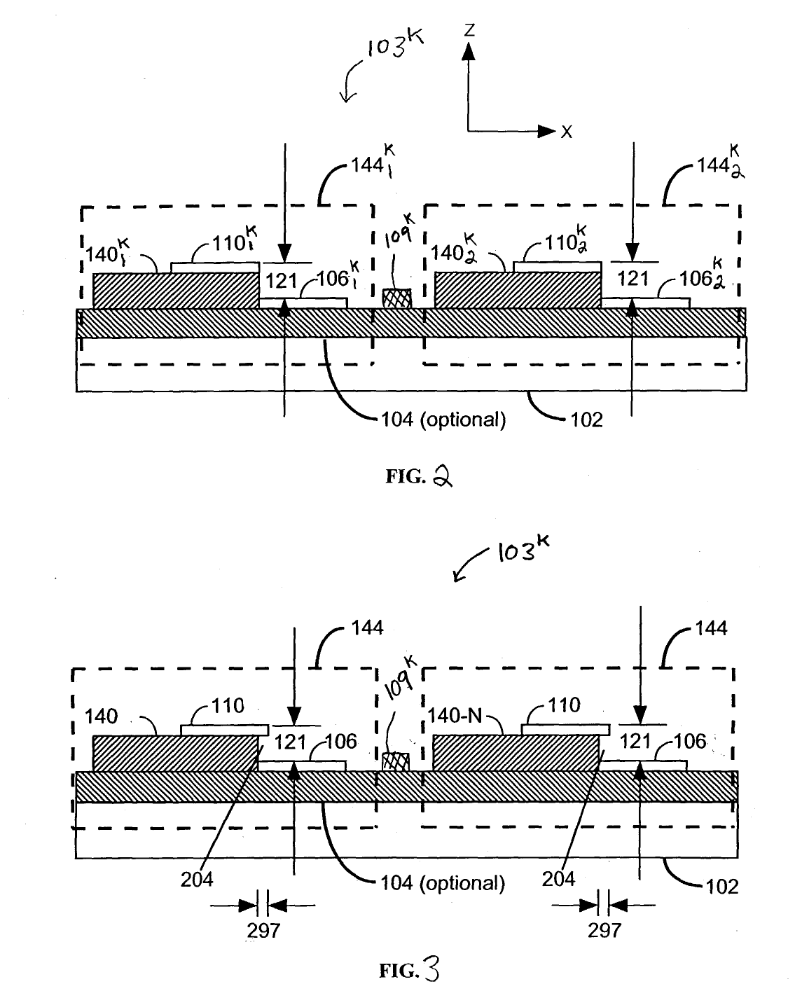

Referring to FIG. 3, a side plan view of the kth subset 103^{k} of sensing devices 144 of a biosensor 200 in accordance with another embodiment of the present invention is shown. Sensing devices 144 of biosensor 200 are similar to sensing devices 144 of FIG. 2, with the exception that materials 106 and 110 overlap each other. As illustrated in FIG. 3, materials 106 and 110 overlap, thereby creating a cavity 204. Furthermore, in the embodiment illustrated in FIG. 3, there is no composition, such as spacer 140 or insulator layer 104 in cavity 204.

The width 297 of cavity 204 defines the amount that materials 106 and 110 overlap in biosensor 200 (FIG. 3). In some embodiments of the present invention, cavity 204 has a width 297 that is 300 Angstroms or less, 250 Angstroms or less, 200 Angstroms or less, 150 Angstroms or less, 100 Angstroms or less, 50 Angstroms or less, or Angstroms or less.

Preparation of a Surface Modified Array of Electrodes

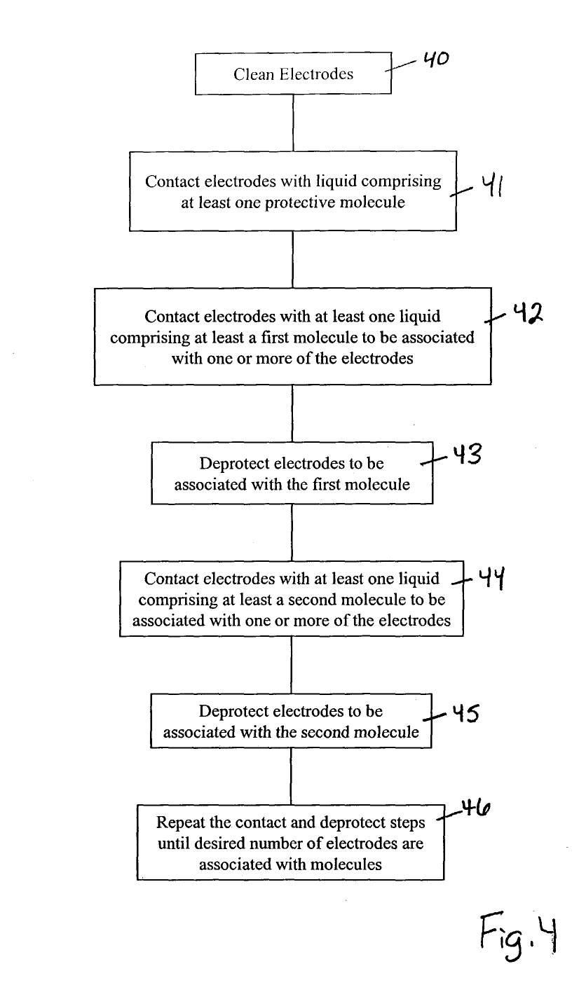

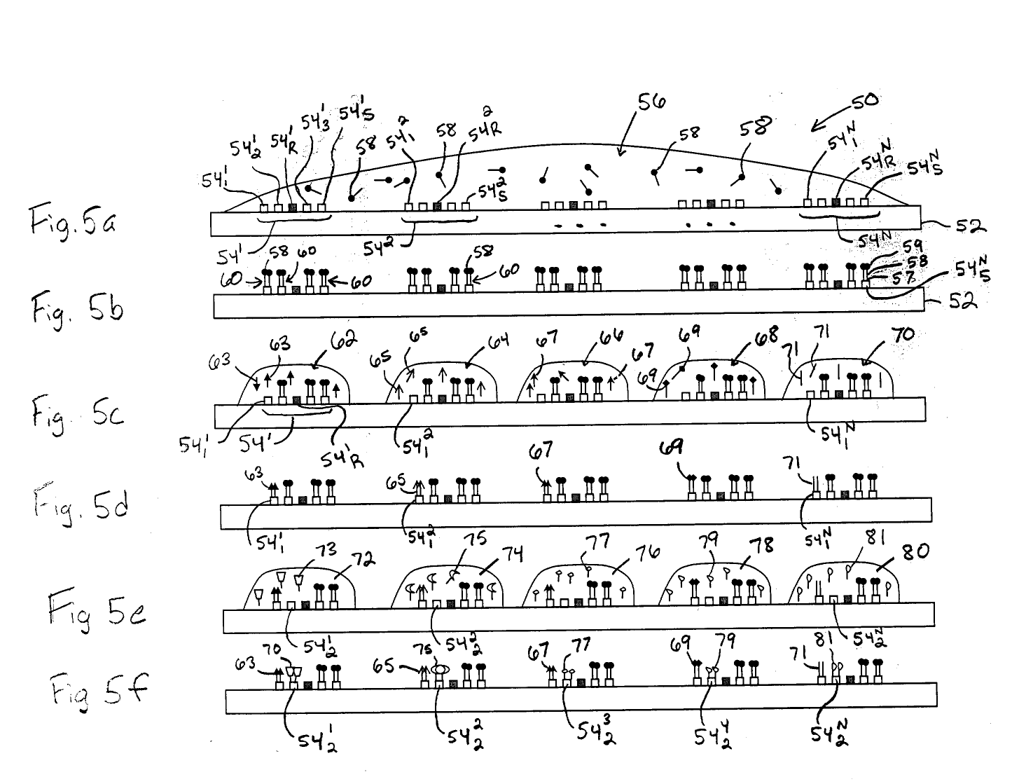

Referring to FIGS. 4 and 5a-5f, one aspect of the present invention relates to the association of molecules with electrodes of an array of electrodes. Preferably, different molecules are selectively associated with different electrodes of an array of electrodes. The association preferably occurs through a covalent bond between the molecule and the electrode. The array may comprise a plurality of independent electrodes, a plurality of electrode pairs, or a combination thereof. The member electrodes of a pair of electrodes operate in conjunction with one another, e.g., through the formation of an electrical connection therebetween, to determine the presence of a target molecule. Independent electrodes may each independently allow the determination of the presence of a target molecule.

Preferred steps of a method in accordance with the present invention are discussed below in reference to flow chart 39 of FIG. 4. Thus, electrodes of an array of electrodes may be cleaned 40, such as to remove organic contaminants. A protective layer comprising at least one protective molecule may be associated with electrodes of the array, such as by contacting 41 the electrodes with a liquid comprising the at least one protective molecule.

Electrodes associated with a protective layer are contacted 42 with a liquid comprising at least one first molecule to be associated with one or more of the electrodes. In one embodiment, all or substantially all of the electrodes of the array are contacted with a liquid comprising the same first molecule. In another embodiment, subsets of the electrodes are contacted with respective liquids, with each liquid comprising at least one different, first molecule to be associated with one or more electrodes of each subset. The first molecule is preferably a probe molecule.

Electrodes to be associated with the at least one first molecule are deprotected 43 by selectively dissociating the overlying protective layer from these electrodes, thereby allowing the first molecules in the liquid contacting the electrodes to associate with the deprotected electrodes. The protective layer, however, inhibits association of the first molecules with electrodes that have not been deprotected. It should be understood that deprotection step 43 may be performed, for example, prior to contacting step 42 or concurrently with contacting step 43.

Once the first molecules have been associated with the deprotected electrodes, the electrodes may be contacted 44 with a liquid comprising at least one second molecule, which may be a different probe molecule. As with contacting step 42, all or substantially all of the electrodes of the array may be contacted with a liquid comprising the same second molecule or combination of second molecules. Alternatively, subsets of the electrodes may be contacted with respective liquids, with each liquid comprising at least one respective, different second molecule to be associated with one or more electrodes of each subset. The number of subsets of electrodes contacted with respective liquids in step 44 may be, but is not required to be, the same as the number of subsets of electrodes contacted with respective liquids in step 42.

Electrodes to be associated with the at least one second molecule are deprotected 45 by selectively dissociating the protective layer from these electrodes, thereby allowing the second molecules in the liquid contacting the electrodes to associate therewith. The protective layer, however, inhibits association of the second molecules with electrodes that have not been deprotected. Deprotection step 45 may be performed, for example, prior to contacting step 44 or concurrently with contacting step 44.

The electrodes to be associated with the at least one second molecule are preferably different from the electrodes associated with the at least one first molecule. The association of the first molecules with an electrode, however, inhibits association of second molecules with these electrodes. Thus, upon the completion of contact step 44, at least two electrodes of the array are associated with different molecules.

The steps of contacting electrodes with a liquid comprising at least one molecule to be associated with at least one electrode of the array and deprotecting electrodes to be associated with the at least one molecules are repeated 46 until a predetermined number of the electrodes have been associated with one or more molecule. Thus, the present invention allows the preparation of an array of electrodes in which each of a plurality of the electrodes is associated with a respective, different electrode. The preparation of such an array of electrodes is discussed in greater detail below.

As seen in FIGS. 5a-5f, an electrode array 50 comprises a substrate 52 comprising a number N^{a} electrodes 54ᵢ. The number Na is an integer, preferably greater than 2, such as greater than 100, for example, greater than 1000, or even greater than 10,000. The number Na comprises electrodes of the array to be modified with a probe molecule but does not comprise reference electrodes that may be used in preparation of the array but are not themselves modified with a probe molecule.

Electrodes 54ᵢ of electrode array 50 may be grouped in subsets 54^{k} of electrodes, where the electrodes within each subset have an index i, where i=1, 2, 3, . . . S, and the subsets have an index k, where k=1, 2, 3, . . . N^{a}. The number of electrodes S within the subsets may be different for different subsets of electrodes. For each subset, however, the number of electrodes S is preferably at least 2, for example, at least 4, such as greater than 10, or even greater than 50. For each subset, the number of electrodes S is preferably less than 1000, such as less than 500 or less than 100, for example, less than 25. A subset comprising fewer than a number N^{a} electrodes is defined herein as a proper subset of electrodes. Thus, for a proper subset of electrodes, S is less than N^{a}.

Each electrode 54i preferably has an electrode surface comprising a conductive material. Suitable conductive materials comprise Au, Pd, Pt, Ag, Cr, Hg, Fe, Cu, Al, Ti, and alloys comprising these materials, such as Au/Pd, Au/Ag, Ag/Pd, GaAs. Other conductive materials, such as doped semiconductors and other conductive or semiconductive inorganic or organic materials, such as 7,7′,8,8′-tetracyanoquinonedimethane (TCNQ), may also be used. Each electrode subset 54^{k} preferably, but not essentially, comprises a reference electrode 54_{R}. The reference electrodes may be any reference electrode generally used in electrochemistry. A preferred reference electrode is a Ag/AgCl, which may be used with or without a salt bridge.

In accordance with the present invention, surfaces of electrodes 54ᵢ of electrode array 52 may be modified to comprise an associated probe molecule, which is typically a molecule, such as an enzyme, receptor, nucleic acid, polynucleotide, protein, lectin, or antibody. For example, a polynucleotide able to hybridize with a second, at least partially complementary polynucleotide, is a preferred probe molecule. With reference to FIGS. 5a-5f, the following discussion describes a method of the invention.

Prior to associating a molecule with electrodes of the array, the electrodes are preferably cleaned 40 to remove surface contaminants. Electrodes 54ᵢ may be cleaned by, for example, contacting the electrodes with an oxidizing material such as a solution comprising between 50% and 80% sulfuric acid and between 50% and 20% hydrogen peroxide. Electrode surfaces may also be cleaned by exposure to ultraviolet light and/or ozone.

Referring to FIGS. 5a and 5b, electrodes 54ᵢ of electrode array 52 are provided with an overlying protective layer 60 comprising at least one protective molecule 58. As defined herein, the term protective layer refers to an amount of protective molecules sufficient to inhibit the association, e.g., binding, of other molecules with the electrode. Each protective layer 60 preferably comprises at least a monolayer comprising at least one protective molecule 58. A protective layer 60, however, may comprise less than a complete monolayer or may comprise more than one layer of protective molecules associated with an electrode. Additionally, any protective layer in accordance with the present invention may comprise more than one type of molecule.

Protective layers in accordance with the present invention may be prepared by contacting 41 electrodes 54ᵢ of electrode array 52 with a liquid comprising a protective molecule 58. Exemplary protective molecules comprise, but are not limited to, alkylsiloxanes, alkanethiolates, and fatty acids. For example, a preferred protective molecule has a structure X—R—Y, where X is a sulfur group, e.g., SH, SPO₃—, OSO₃H, Z-S—S-(where Z is an alkyl group, such as an alkane), R comprises a linear or branched alkyl group, which is preferably an alkane, and Y may be selected from the group comprising hydrogen, alcohols, carboxylic acids, esters, alkenes, ketones, aldehydes, amines, sulfonic acids, halogens, and alkyl halogens. Protective molecules comprising a sulfur group, such as a thiol, a thioate, a sulfide, or alkylthiolate, are preferred especially where electrodes 54ᵢ comprise a gold or platinum surface. The sulfur group may bind with the electrode.

Preferred protective molecules may comprise a first portion that associates with an electrode and a second portion disposed to inhibit the association of other molecules with an electrode having a protective layer of the protective molecules. For example, referring to FIG. 5b, a protective molecule 58 associated with an electrode 54_{S}^{N} of subset 54^{N} comprises a first portion 57 and a second portion 59. First portion 57 is associated, such as by a covalent bond, with electrode 54_{S}^{N}. Second portion 59, which may be a terminus of the protective molecule, is preferably spaced apart from first portion 57 and from electrode 54_{S}^{N}, Second portion 59 is thereby exposed to molecules present in a liquid contacting the electrode. Thus, the physio-chemical characteristics of the second portion 59 may be varied, such as by comprising groups having different charges and hydrophobicities, to optimize the protective function of a protective layer 60. For example, a protective molecule comprising a hydrophobic second portion may be used to inhibit hydrophillic molecules from associating with an electrode surface. The protective molecules may be selected from, for example, at least one of an alcohol, a carboxylic acid, an ester, an alkane, an alkene, a ketone, and aldehyde. Second portion 59 may also comprise chemical groups, such as —CHₓR_{y}, —OH, —(C═O)OCHₓR_{y}, —COOHₓ, and —OSO₃Hₓ, where x is between 0 and 3, R is halogen, and y is between 0 and 3.

In an exemplary embodiment, electrodes 54ᵢ of electrode array 52 are provided with an overlying protective layer by contacting the electrodes with a liquid 56 comprising an alkylthiolate, such as mercaptohexanol, preferably under conditions suitable to associate a self-assembled monolayer of the alkylthiolate with electrodes 54ᵢ. For example, the liquid may be an aqueous solution comprising at least 250 μM, such as at least 500 μM of the alkylthiolate. The aqueous solution may comprise less than 10 mM alkylthiolate, such as less than 5 mM. Liquid 56 comprising the protective molecule is contacted with electrodes 54ᵢ for a time sufficient to prepare a protective layer 60 that inhibits other molecules from associating with electrodes having the protective layer. For example, electrodes 54ᵢ may be exposed to liquid 60 for at least 15 minutes, such as at least 30 minutes. Electrodes 54ᵢ may be exposed to liquid 60 for less than 300 minutes, such as less than 150 minutes. Molecules 58 of the protective layer are preferably covalently associated with the electrodes, such as through a covalent bond between a sulfur group of the protective molecule and the electrode surface. Following exposure to the protective molecules, electrodes of the array may be contacted with a liquid, such as ethanol or other solvent, to remove any protective molecules not covalently associated with an electrode.

Preferably after forming a protective layer 60, electrodes 54ᵢ to be associated with one or more first molecules may be contacted 42 with a liquid comprising the first molecule. As seen in FIG. 5c, respective subsets 54^{k} of electrodes 54ᵢ may be contacted with respective liquids, with each liquid preferably comprising at least one different, first molecule to be associated with at least one electrode of a respective subset. For example, electrode subset 54¹ is contacted with a liquid 62 comprising a molecule 63, electrode subset 54² is contacted with a liquid 64 comprising a molecule 65, electrode subset 54³ is contacted with a liquid 66 comprising a molecule 67, electrode subset 54³ is contacted with a liquid 68 comprising a molecule 69, and electrode subset 54^{N} is contacted with a liquid 70 comprising a molecule 71. Each of molecules 63, 65, 67, 69, and 71 may be a different probe molecule, e.g., a polynucleotide comprising a different sequence.

The liquid that contacts the electrodes of a subset preferably also contacts a reference electrode, thereby electrically contacting the electrodes of a subset and the reference electrode. For example, liquid 62 contacts electrodes 54ᵢ¹ of electrode subset 54¹ and reference electrode 54_{R}¹. Similarly, liquid 64 contacts electrodes 54ᵢ² of electrode subset 54² and reference electrode 54_{R}². Preferably, the liquids contacting electrodes of different subsets of electrodes do not establish electrical contact between the electrodes of different subsets. For example, electrodes 54ᵢ¹ may be electrically isolated from electrodes 54ᵢ² despite the presence of liquids 62 and 64, which liquids contact different regions of substrate 52. Thus, the electrical potential of electrodes 54ᵢ¹ may be modified with respect to reference electrode 54_{R}¹ independently of an electrical potential difference between electrodes 54ᵢ² reference electrode 54_{R}².

Liquids may be applied to respective subsets of electrodes in the form of, for example, droplets or as a liquid flow. The liquids applied to different subsets of electrodes may be identical except for the presence of different molecules therein. Alternatively, different subsets of electrodes may be contacted with different liquids, such as different solvents and/or similar solvents having different ionic strengths. In any event, the liquid is preferably an electrolyte, such as an electrolyte solution, which may comprise, for example, an aqueous solution of electrolytes, an organic electrolyte solution of electrolytes, and mixtures thereof.

Upon contacting a plurality of electrodes with a liquid comprising at least one first molecule to be associated with one or more of the electrodes, the electrodes to be associated with the first molecule are deprotected 43 by dissociating the protective layer from these molecules. Deprotection of an electrode preferably comprises modifying an electrical potential of an electrode or an electrical potential difference between the electrode and a reference electrode, whereby the protective layer 60 disassociates from the electrode allowing other molecules to associate with the electrode. For example, modifying an electrical potential difference between electrode 54₁¹ and reference electrode 54_{R}¹ causes the protective layer 60 associated with electrode 54₁¹, to dissociate therefrom. Molecules 58 of protective layer 60 may dissociate by diffusing away from the electrode and/or under by moving under the influence of an electric field, such as an electric field formed between electrode 54₁¹ and reference electrode 54_{R}¹. Dissociation preferably comprises breaking a covalent bond, e.g., a covalent bond between a sulfur group of the protective molecule and the electrode surface. Similarly, protective layer 60 dissociates from electrode 54₁² upon modifying an electric potential difference between electrode 54₁² and reference electrode 54_{R}². Protective layer 60 dissociates from electrode 54₁^{N} upon modifying an electrical potential difference between electrode 54₁^{N} and reference electrode 54_{R}^{N}.

To deprotect an electrode, the electrical potential difference between the electrode and a reference electrode is preferably sufficient to cause reduction of protective molecules associated with the electrode surface and subsequent dissociation therefrom. For example, in one embodiment the protective molecules may be associated with a gold electrode surface through a covalent sulfur bond. The electrodes are contacted with a liquid having a pH of between 4 and 10, such as between 5 and 8, and the electrodes are deprotected by applying a potential of less than −250 mV, such as less than −500 mV, for example less than −1200 mV, with respect to a Ag/AgCl reference electrode. The composition of the protective layer determines the electrical potential difference and necessary to achieve deprotection. Varying the duration for which the electrical potential is modified allows further control over the degree of protective layer dissociation to be controlled. Upon modifying the electrical potential, the sulfur group of the protective molecule is reductively desorbed according to the reaction:

—SAu(absorbed)+e−—>S⁻+Au

where —SAu represents a protective molecule comprising a sulfur group associated with a gold electrode surface and —S⁻ represents the dissociated, reduced protective molecule. Only electrodes for which the electrical potential has been modified will be deprotected by dissociation of the protective layer.

Once the protective layer has dissociated from an electrode, other molecules present in a liquid contacting the electrode may associate with the electrode. For example, molecules 63 of liquid 62 associate with electrode 54₁¹, which has been deprotected as described above. Molecules 63, however, are inhibited by protective layers 60 from associating with electrodes 54₂¹, 54₃¹, and 54_{S}¹ of subset 54¹. Similarly, molecules 65 of liquid 64 associate with electrode 54₁² Molecules 65, however, are inhibited by protective layers 60 from associating with electrodes 54₂², 54₃², and 54_{S}² of subset 54². In accordance with contacting step 42 and deprotection step 43, one or more different, first molecules may be associated with respective electrodes of different subsets of electrodes. Following exposure of electrodes to the molecules, electrodes of the array may be contacted with a liquid, such as ethanol or other solvent, to remove any molecules not covalently associated with an electrode.

Following the association of a first molecule with at least one electrode of the array, electrodes of array 52 may be contacted 44 with liquid comprising at least one second molecule, e.g., a second probe molecule, to be associated with other electrodes of the array. As seen in FIG. 5e, respective subsets 54^{k} of electrodes 54ᵢ may be contacted with respective liquids, with each liquid preferably comprising at least one different, second molecule to be associated with at least one electrode of a subset. For example, electrode subset 54¹ is contacted with a liquid 72 comprising a molecule 73, electrode subset 54² is contacted with a liquid 74 comprising a molecule 75, and electrode subset 54^{N} is contacted with a liquid 80 comprising a molecule 81.

Although the member electrodes of subsets 54^{k} shown in FIG. 5e correspond to the members of electrode subsets 54^{k} seen in FIG. 5c, subsets of electrodes having a different number of member electrodes may be contacted with respective liquids in different contacting steps in accordance with steps 42, 44 and 46 of FIG. 4. For example, the number of member electrodes of each subset may be determined by the fluid contacting the electrodes rather than organization of electrodes and reference electrodes within array 50.

Once electrodes have been contacted with a liquid comprising one or more second molecules, as seen in FIG. 5e, electrodes to be associated with the second molecules are deprotected 45. Deprotection is preferably performed as described above. Thus, for example, electrode 54₂² of subset 54² is deprotected by modifying an electrode potential between electrode 54₂² and reference electrode 54_{R}², thereby allowing molecules 75 to associate with the deprotected electrode. Similarly, for example, electrode 54₂³ of subset 54³ is deprotected by modifying an electrode potential between electrode 54₂³ and reference electrode 54_{R}³ thereby allowing molecules 77 to associate with the deprotected electrode.

The steps of contacting 44 electrodes with a liquid comprising a molecule to be associated with an electrode and deprotecting 45 selected electrodes are repeated until electrode in the array has been associated with a predetermined molecule. For example, for exemplary array 50, in which each subset comprises 4 electrodes, the contacting and deprotecting steps would be repeated a total of 4 times to associate each electrode with a different molecule.

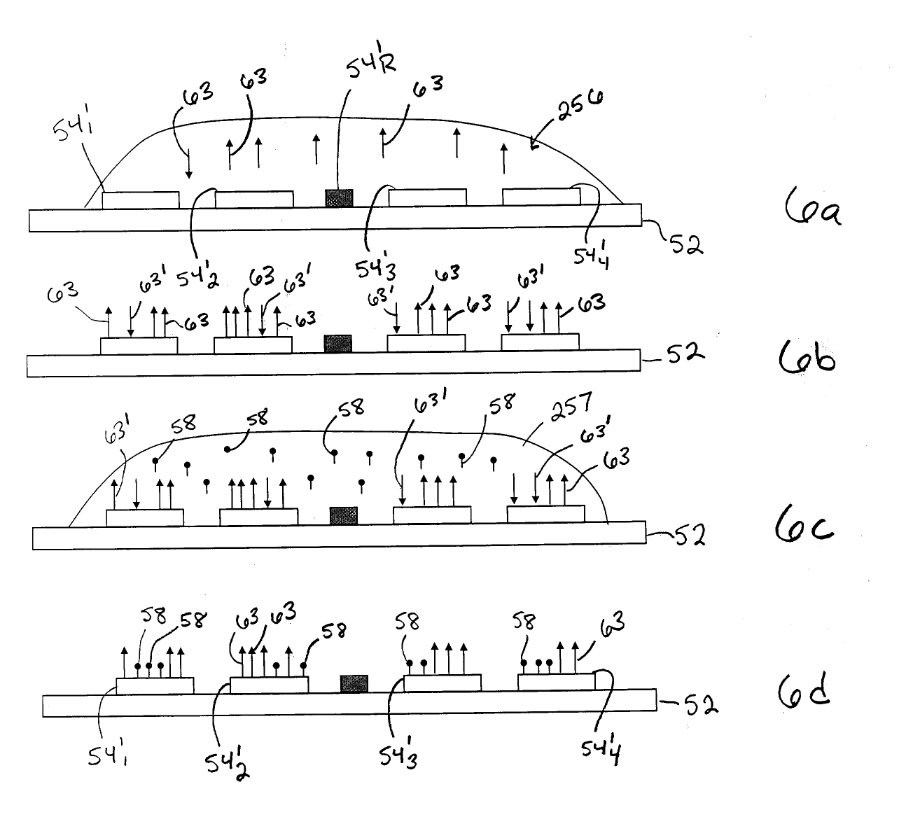

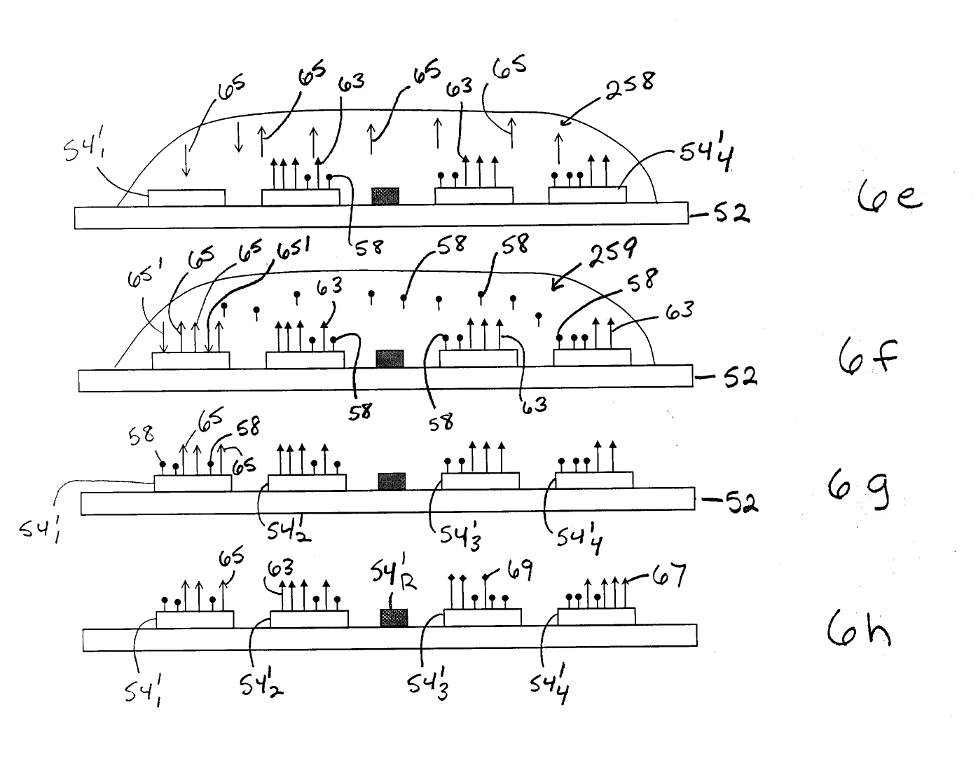

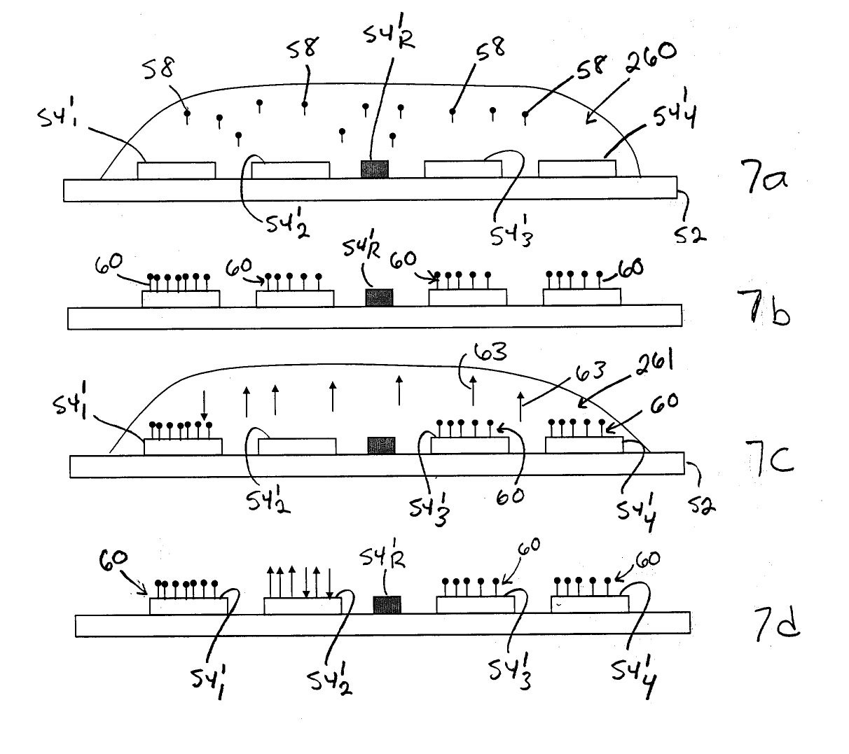

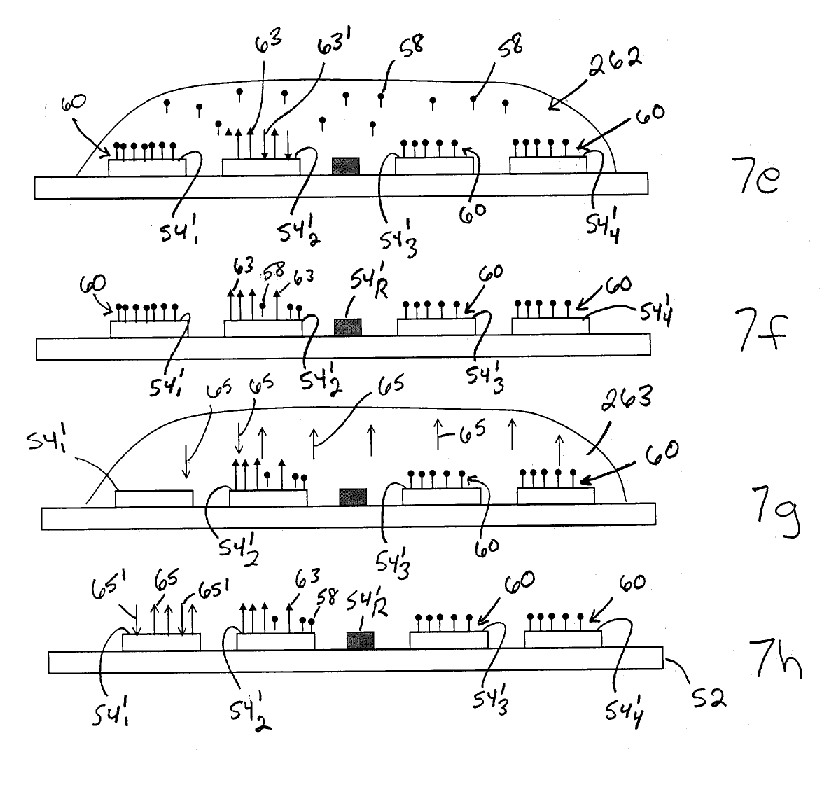

Referring to FIGS. 6a-6h, an embodiment of a method for preparation of an array of modified electrodes is illustrated in which electrodes of the array are not first provided with an overlying layer 60 of protective molecules 58 prior to contacting electrodes with a liquid comprising a probe molecule. FIGS. 6a-6h show only a single subset 54¹ of electrodes 54ᵢ of array 50. It should be understood, however, that steps of the method may be applied to more than one subset of electrodes as discussed above with reference to FIGS. 5a-5f.

As seen in FIG. 6a, electrodes of subset 54¹ are contacted with a liquid 256 comprising a probe molecule 63. Electrodes may be cleaned prior to or in conduction with being contacted with liquid 256 but are not overlaid with one or more protective molecules prior to being contacted with liquid 256. Probe molecules 63 associate, such as by covalently binding, with electrodes 54ᵢ. Some probe molecules 63′, however, may exhibit non-specific association, which refers to association with electrode by other than covalent bonds. The presence of non-specifically associated probe molecules is undesirable because different probe molecules intended to be bound to other electrodes in a subsequent contacting step may displace non-specifically bound probe molecules previously associated with electrodes of the array. The different probe molecules, therefore, may undesirably bind to electrodes to which the different probe molecules were not intended to bind. Such undesired binding may reduce the specificity of the array if electrodes of the array are made at least partially sensitive to the presence of more than one different molecule.

Referring to FIGS. 6c and 6d, electrodes of the array are contacted with a liquid 257 comprising a protective molecule 58, which may be any protective molecule in accordance with the invention. Protective molecule 58 displaces non-specifically associated probe molecules 63′ from electrodes 54ᵢ of the array thereby preparing electrodes having both probe molecules 63 and protective molecules 58 bound thereto (FIG. 6d). Protective molecule 58 is preferably shorter than probe molecules to be bound to electrodes of the array so that the protective molecules will not sterically hinder the association between a target molecule and a probe molecule bound adjacent a protective molecule. For example, protective molecules having a formula HS—(CH₂)ₓ—Y, where x at least one and less than 15, for example, less than 10, and Y is a functional group, for example, an alcohol, may be used.

Referring to FIG. 6e, electrodes of the array are contacted with a liquid 258 comprising a probe molecule 65, which is different from the probe molecule 63 of the contacting step of FIG. 6a. The previously bound probe molecules 63 and protective molecules 58 inhibit the different probe molecule 65 from associating with electrodes 54ᵢ of the array. However, one of the electrodes of the array, here 54₁, may be subjected to a deprotection step in which molecules associated with the electrode, such as through a covalent bond, are dissociated from the electrode. Thus, probe molecules 63 and protective molecules 58 associated with the electrode 54₁ in previous contacting steps dissociate from the electrode.

The deprotection step is preferably performed by modifying an electrical potential of the electrode in accordance with step 43 of flow chart 39. Alternatively, the deprotection may be performed by modifying an electrical potential difference between the electrode and a reference electrode. For example, the electrode may be electrically addressed to modify an electrical potential of the electrode or modify an electrical potential difference between the electrode and a reference electrode. The deprotection step may be performed prior to contacting the electrodes with a liquid having a probe molecule or concurrently therewith. If deprotection is performed concurrently with the step of contacting the electrode with a molecule to be bound to the electrode, it is preferred that the electrode is not concurrently subjected to a modified electrical potential or electrical potential difference during the entire time that the liquid is in contact with the electrode.

Referring to FIG. 6f, the different probe molecule 65 associates with the electrode 54, from which the previously overlying probe molecules 63 and protective molecules 58 were dissociated. A first portion of the different probe molecules 65 may associate with the electrode by covalent binding while a second portion 65′ may associate with the electrode through non-specific association. The electrodes 54ᵢ of the subset are contacted with a liquid 259 comprising a protective molecule 58, which displaces non-specifically associated probe molecules 65′ from electrode 54, (FIG. 6g).

Referring to FIG. 6g, subset 54¹ of electrodes 54ᵢ is shown after having been contacted to a total of two cycles in accordance with the method. Each cycle comprises (i)contacting electrodes of the subset with a liquid comprising a probe molecule, (ii) contacting electrodes of the subset with a liquid comprising a protective molecule, and (iii) dissociating previously bound probe molecules and protective molecules from at least one of the electrodes. During each cycle, therefore, a protective molecule and a different probe molecule may be overlaid on a respective electrode. The protective molecules used in each cycle may be different or may be the same. As seen in FIG. 6h, the subset of electrodes may be subjected to a number of cycles equal to the number of electrodes S in the subset to thereby prepare a subset of electrodes in which each electrodes is modified with a different probe molecule.

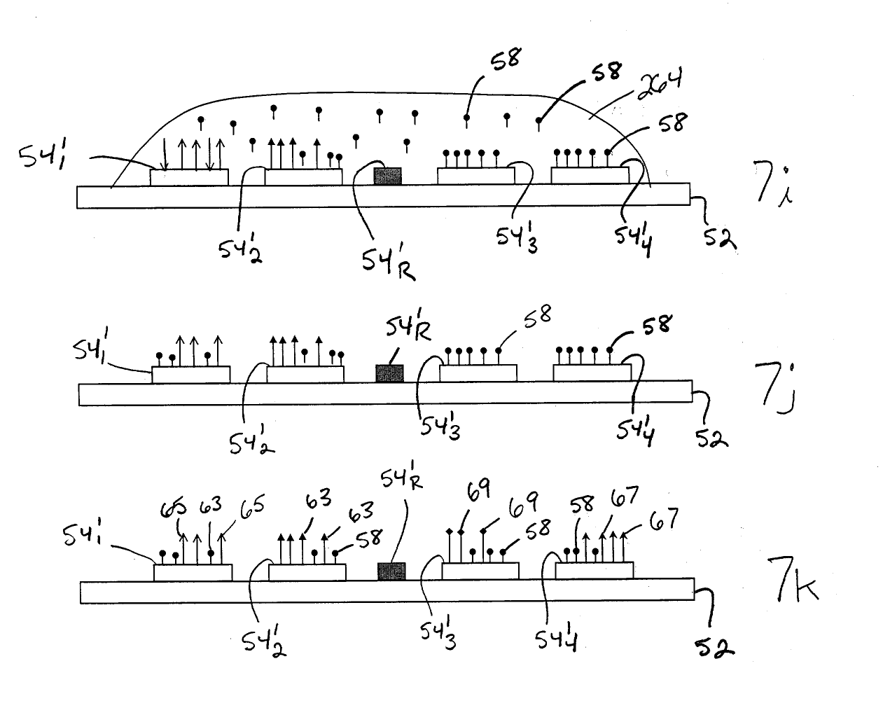

Referring to FIGS. 7a-7k, an embodiment of a method for preparation of an array of modified electrodes is illustrated in which electrodes of the array are first provided with an overlying layer 60 of protective molecules 58 prior to contacting electrodes with a liquid comprising a probe molecule. Preparation of the array continues in cycles of steps. Each cycle comprises steps of (i) contacting electrodes of the array with a liquid comprising a probe molecule, (ii) contacting electrodes of the array with a liquid comprising a protective molecule, and (iii) deprotecting an electrode not having a probe molecule associated therewith. FIGS. 7a-7k show only a single subset 54¹ of electrodes 54ᵢ of array 50. It should be understood, however, that steps of the method may be applied to more than one subset of electrodes as discussed above with reference to FIGS. 5a-5f.

Referring to FIGS. 7a-7k, electrodes 54ᵢ of the first subset 54¹ of electrodes of array 50 are modified by a method comprising contacting the electrodes of the array with a liquid 260 comprising one or more protective molecules 58 prior to associating a probe molecule with one or more electrodes of the array. (FIG. 7a). As seen in FIG. 7b, the electrodes contacted with liquid 260 are each overlaid with a protective layer 60 comprising protective molecules 58, which may be any protective molecule in accordance with the present invention.

Referring to FIG. 7c, electrodes of the subset are contacted with a liquid 261 comprising a probe molecule 63, which may be any probe molecule in accordance with the present invention. One of the electrodes, 54₂¹, is shown as having been deprotected in accordance with the present invention. Thus, protective molecules 58 have been dissociated from deprotected electrode 54₂¹. Deprotection may be performed prior to contacting electrodes with liquid 260 and probe molecule 63 or may be performed in conjunction therewith.

Referring to FIG. 7d, probe molecules 63 are associated with electrode 54₂¹. A first portion of the probe molecules 63 may be associated by a covalent bond, e.g., through a sulfur group of the probe molecule and the electrode surface. Other probe molecules 63′ may be non-specifically associated with the electrode. To displace non-specifically associated probe molecules 63′, electrodes of subset 54¹ are contacted with a liquid comprising a protective molecule 58, which displaces non-specifically associated probe molecules 63′ from electrode 54₂¹. Subsequently, electrode 54₂¹ has both probe molecules and protective molecules bound thereto. (FIG. 7f).

Referring to FIG. 7g, electrodes of the subset are contacted with a liquid 263 comprising a probe molecule 65. Another one of the electrodes, 54₁¹, is shown as having been deprotected in accordance with the present invention. Thus, protective molecules are dissociated from deprotected electrode 54₁¹. Probe molecules 65 may associate with electrode 54₁¹ both by covalent binding and by and non-specific association. (FIG. 7h).

Referring to FIG. 7i, electrodes of the subset 54¹ are contacted with a liquid 264 comprising a protective molecule 58, which displaces non-specifically associated probe molecules 65′ from electrode. The result, as seen in FIG. 7j, is that probe molecule 65 and protective molecules 58 are bound to electrode 54₁¹ and probe molecule 63 and protective molecules 58 are bound to electrode 54₂¹. Protective molecules 58 associated with electrodes 54₃¹ and 54₄¹ inhibit probe molecules from associating with these electrodes.

Referring to FIG. 7k, subset 54¹ of electrodes 54ᵢ is shown after having been contacted to a total of four cycles in accordance with the method. Each cycle comprises (i) contacting electrodes of the subset with a liquid comprising a probe molecule, (ii) contacting electrodes of the subset with a liquid comprising a protective molecule, and (iii) deprotecting at least one electrode by dissociating protective molecules from the at least one electrode. During each cycle, therefore, a protective molecule and a different probe molecule may be overlaid on a respective electrode. The protective molecules used in each cycle may be different or may be the same.

Preparation of an Array of Surface Modified Electrode Pairs

One aspect of the present invention relates to a method for preparing a biosensor comprising a plurality of surface modified electrode pairs, which may be used to determine the presence of one or polynucleotides. Methods for preparing an array of modified electrode pairs are discussed generally below and then in detail with reference to FIGS. 6a-6e.

A method for preparing a biosensor comprising a plurality of surface modified electrode pairs comprises associating a molecule with a first electrode of an electrode pair. The first molecule is preferably a probe molecule comprising a first polynucleotide, which is preferably single stranded. A protective molecule may be also associated with the first electrode to displace non-specifically associated first molecules, as discussed above with reference to FIGS. 6a-6h and 7a-7k. A second molecule is associated with the second electrode of the electrode pair. The second molecule comprises a group configured to preferentially associate with double stranded polynucleotides. For example, the second molecule comprise an intercalating group or a grove binder. A protective molecule may be also associated with the second electrode to displace non-specifically associated second molecules, as discussed above with reference to FIGS. 6a-6h and 7a-7k.

If the biosensor comprises an array of electrode pairs, different first molecules may be associated with an electrode of other electrode pairs of the array. Second molecules, comprising an intercalating group, may be associated with the other electrode of the other electrode pairs.

To determine the presence of a target polynucleotide, electrode pairs having associated first and second molecules are contacted with the target polynucleotide, preferably by contacting the electrode pairs with a liquid comprising the target polynucleotide. The target polynucleotide is preferably single stranded. If the target polynucleotide is at least partially complementary to a first polynucleotide of a first molecule associated with an electrode of an electrode pair, the first and second polynucleotides may form a duplex region, such as by at least portions of the polynucleotides annealing. The group of the second molecule associated with the other electrode of the electrode pair associates with the duplex region, thereby modifying an electrical characteristic of the first and second electrodes. For example, the second molecule may comprise an intercalating group that intercalates with the duplex region, thereby forming an electrical connection between the first and second electrodes. The electrical connection reduces an electrical resistance between the first and second electrodes of the pair.

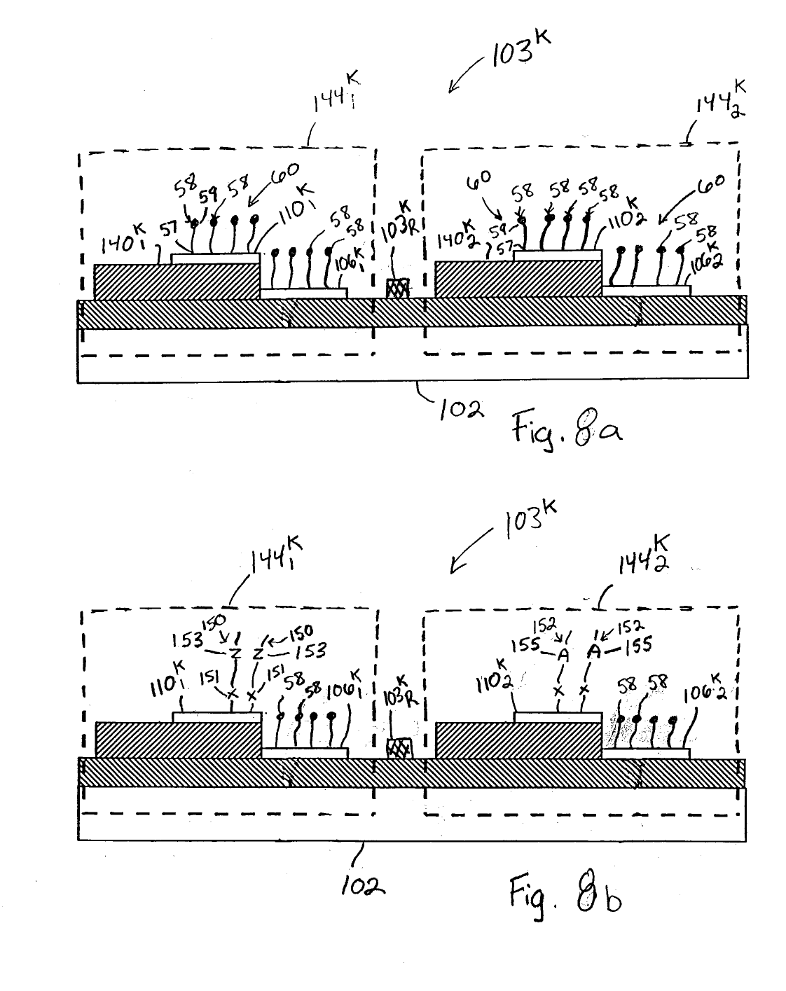

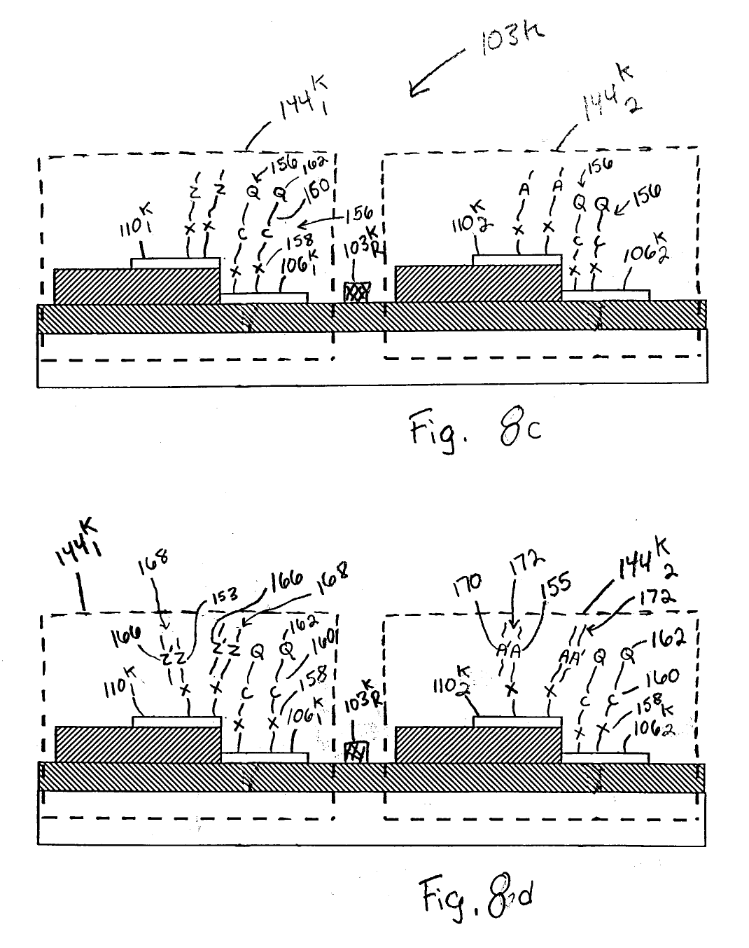

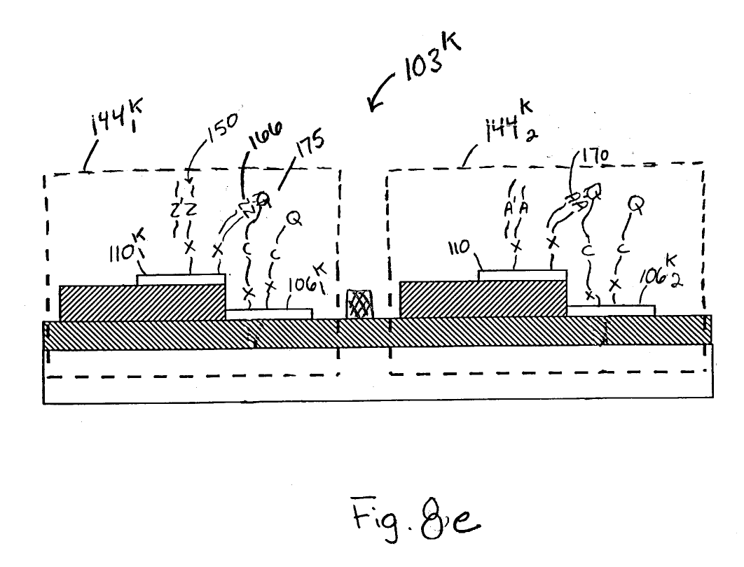

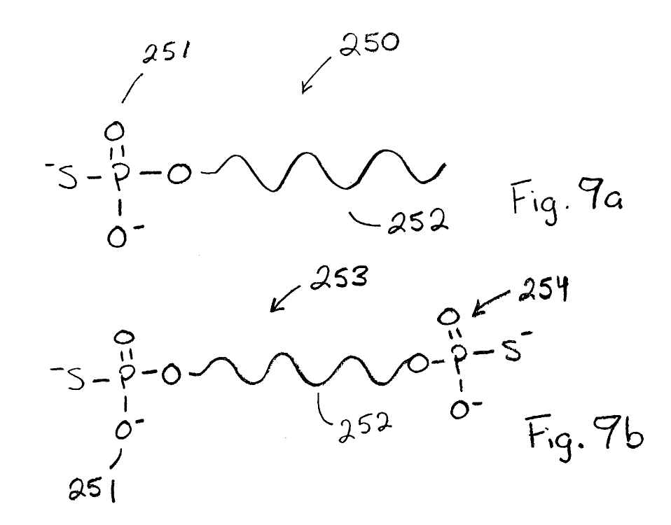

Referring to FIG. 8a, a cross-sectional side view of the kth subset 103^{k} of sensing devices 144 of biosensor 100 is shown. As discussed above, each device comprises an electrode pair. Each electrode 106 and 110 of the electrode pairs 144₁^{k} and 144₂^{k} is preferably independently addressable, such as by a voltage or current source, preferably so that a voltage or electrical current may be applied independently to any desired electrode of biosensor 100. For example, a voltage or current applied to an electrode 110₁^{k} may be modified independently of a voltage or current associated with other electrodes of biosensor 100.

In accordance with step 41 of flow chart 39, a layer 60 of protective molecules 58 is associated with each electrode, preferably by contacting electrode pairs of the biosensor with a liquid comprising one or more protective molecules 58. Layer 60 of protective molecules 58 inhibits the association of other molecules with a protected electrode. Protective molecules 58 may be any protective molecule in accordance with the present invention. Thus, protective molecules 58 preferably comprise a first portion 57, which associates with a protected electrode, and a second portion 59, which is exposed to molecules present in a liquid contacting the protected electrode. In combination with or prior to preparing the protective layer, the electrodes of the array may be cleaned to remove organic contaminants.

Referring to FIG. 8b, the method for preparing an array of modified electrode pairs continues by associating a first molecule with a first electrode of each electrode pair. Each first molecule comprises first and second portions. The first portion comprises a group that may be associated with a surface of an electrode. Preferred groups comprise sulfur. The second portion of each first molecule preferably comprises a polynucleotide, e.g., a single stranded polynucleotide. First molecules to be associated with different electrodes preferably comprise polynucleotides having different sequence so that the different first molecules will hybridize with different single stranded polynucleotides. A protective molecule 58 may be also associated with the first electrode to displace non-specifically associated first molecules, as discussed above with reference to FIGS. 6a-6h and 7a-7k.

Referring to FIG. 9a, an exemplary first molecule 250 comprises a single stranded polynucleotide 252 having an unprotected phosphorothioate group 251 associated with, for example, the 3′ end of the molecule. A phosphorothioated polynucleotide is a polynucleotide in which at least one oxygen of at least one of the phosphate groups of the polynucleotide is replaced by sulfur. By unprotected, it is meant that a sulfur of the phosphorothioate group is available to bind with a surface, such as the gold surface of an electrode. In some embodiments, the first molecule comprises only a single phosphorothioate group. Only a single oxygen of the phosphorothioate group may be replaced by sulfur. In other embodiments, the first molecule comprises only a single phosphorothioate group in which two oxygens are replaced by sulfur. Thus, unless specified to the contrary, the term phosphorothioate group, as defined herein, is understood to comprise phosphorodithioate groups in which each of at least two of the oxygens have been replaced by sulfur. In either embodiment, it is preferred that only one oxygen of the phosphorothioate group be associated via a chemical bond with a base of a polynucleotide.

Suitable phosphorothioate groups may be synthesized using, for example, chemical synthesis, e.g., comprising use of a sulfurizing reagent in an oxidation step, or by enzymatic incorporation. Suitable synthetic techniques are disclosed in U.S. Pat. No. 5,003,097 to Beaucage et al., which is incorporate herein. Chemical synthesis may comprise introducing a terminal phosphate modification followed by oxidization and sulfurization using, for example, iodine and Beaucage's reagent.

In some embodiments, as with molecule 250, one of the ends of the single stranded polynucleotide, e.g., the 5′ end, is unmodified so that the first molecule may hybridize with other single stranded polynucleotides that are complementary to at least a portion of the first molecule. In other embodiments, as shown for a molecule 253, both the 3′ and 5′ ends of the polynucleotide are phosphorothioated. (FIG. 9b). Molecule 253 may bind covalently with a surface via phosphorothioate group 251 and via a phosphorothioate group 254.

Returning to FIGS. 8a-8e, the association of the first molecules and the electrodes is preferably performed in accordance with step 42 of flow chart 39, e.g., by contacting electrode pairs of the array with at least one liquid comprising at least one first molecule to be associated with an electrode of at least one electrode pair. A first molecule present in the liquid contacting an electrode may be associated with the electrode by deprotecting the electrode in accordance with step 44 of flow chart 39. Thus, electrode deprotection is preferably performed by modifying an electrical potential of an electrode with respect to a reference electrode, as described above. For example, referring to FIG. 8b, sensor devices 144₁ and 144₂ of subset 103^{k} may be contacted with a liquid comprising a molecule 150, which comprises a first polynucleotide 153. Upon modifying an electrical potential between electrode 110, and reference electrode 103_{R}^{k}, protective layer 60 dissociates from electrode 110₁, thereby allowing molecule 150 to associate with the electrode, preferably via a first portion 151 of the molecules. Because other electrodes of subset 103^{k} have not been deprotected, molecules 150 are inhibited from associating with the other electrodes.

In a second contacting step, sensor devices 144₁ and 144₂ of subset 103^{k} may be contacted with a liquid comprising a molecule 152. Molecule 152 preferably comprises a first polynucleotide 155, which is preferably different from first polynucleotide of molecule 150. Upon modifying an electrical potential between electrode 110₂ and reference electrode 103_{R}^{k}, protective layer 60 dissociates from electrode 110₂, thereby allowing molecule 152 to associate with the electrode, preferably via a first portion 152 of the molecules. Because other electrodes of subset 103k have not been deprotected, molecules 152 are inhibited from associating with the other electrodes. The presence of molecules 150 inhibits molecules 152 from associating with electrode 110₁.

As discussed above with reference to FIGS. 5a-5f, other subsets of electrode pairs of biosensor 100 may be contacted with respective liquids, each liquid comprising at least one different molecule to be associated with an electrode of an electrode pair of the subset of electrode pairs. Thus, for example, during the periods of time in which the electrode pairs of subset 103^{k} are contacted with the respective liquids comprising molecules 153 and 155, other subsets of electrode pairs may be contacted with liquids comprising different molecules to be associated with electrodes of the other electrode pairs.

The steps of contacting and deprotecting first electrodes of the electrode pairs of the array may be repeated until each first electrode is associated with a different first molecule. A protective molecule may also be associated with each first electrode. By contacting subsets of electrode pairs with respective liquids, each comprising a respective different first molecule, a plurality of different electrodes may each be associated with a different first molecule during each cycle of contacting and deprotecting. Therefore, the present invention allows an array comprising a plurality of electrode pair array, each associated with a different molecule, to be prepared in less time than would be required to contact all electrode pairs with a liquid comprising the same molecule and deprotecting only 1 electrode of the array during each contacting step.

Referring to FIG. 8c, a second molecule is associated with second electrodes of the electrode pairs. For example, a molecule 156 is associated with both electrodes 106₁ and 106₂. Electrodes to be associated with a second molecule are preferably contacted with a liquid comprising the second molecule and deprotected to allow dissociation. Because the same second molecule may be associated with the second electrode of each electrode pair of the array, the step of associating the second molecules may be performed in a single step by simultaneously contacting all electrodes of the array with a liquid comprising the same second molecule. Of course, different second molecules may be associated with the second electrodes of different electrode pairs. In such embodiments of the invention, subsets of the electrodes may be contacted with respective liquids each comprising a different second molecule. Only those electrodes to be associated with a particular second molecule are deprotected.

As seen in FIG. 8c, a preferred second molecule comprises a first portion 158, a second portion 160, and a third portion 162. Second molecule 156 preferably associates with electrodes 106 via first portion 158. Thus, first portion 158 comprises any group, e.g., a sulfur group, that may be associated with an electrode, preferably by forming a covalent bond with a surface of the second electrode. For example, first portion 158 may comprise a phosphorothioate, a thiol, a thioate, a sulfide, or an alkylthiolate.

Second portion 160 of second molecule 156 preferably comprises a conductive oligomer. Conductive oligomers are also referred to in the literature as molecular wires and the terms are used synonymously herein. Suitable conductive oligomers are disclosed in U.S. Pat. No. 6,479,240, issued Nov. 12, 2002, to Kayyem et al. and hereby incorporated by reference. Typical conductive oligomers comprise a plurality of monomeric units, which share conjugated π-orbitals, e.g., the conductive oligomer may comprise a plurality of interspersed double and/or triple bonds. Of course, suitable conductive oligomer may also contain one or more σ bonds. Examples of conductive oligomers comprise oligo pheylene vinylene and poly pyrroles.

In preferred embodiments, the conductive oligomer has a length of between 20 and 200 Angstroms and a conductivity, S, of at least 10⁻⁶ Ω⁻¹ cm⁻¹, e.g., at least 10⁵ Ω⁻¹ cm⁻¹. A conductive oligomer may have a conductivity of less than 10⁴ Ω⁻¹ cm⁻¹, e.g., less than 10² Ω⁻¹cm⁻¹. Thus, the rate of electron transfer through preferred conductive oligomers is faster than the rate of electron transfer through double stranded polynucleotides, i.e. through the pi-orbitals of a double helix.

In some embodiments, third portion 162 of second molecule 156 comprises an intercalating group, which is configured to intercalate with double stranded polynucleotides. Preferred intercalating groups preferentially associate with double stranded polynucleotides as compared to single stranded polynucleotides. Exemplary intercalating groups comprise ethidium bromide, acridine, and derivatives of these compounds. Exemplary acridine derivatives comprise acridine orange, acridine yellow, 9-aminoacridine, hydrochloride hydrate, 2-aminoacridone, 9,9′-biacridyl, 9-chloroacridine, 6,9-dichloro-2-methoxyacridine, n-(1-leucyl)-2-aminoacridone, and 10-octadecyl acridine orange. Other suitable intercalators comprise rivanol, doxorubicin, daunorubicin, actinomycin D, 7-amino Actinomycin D, ellipticine, coralyne, propidium, TAS103, berberine, distamycin, berenil, 7H-methylbenzo[e]pyrido[4,3-b]indole, meso-tetrakis(N-methyl-4pyridyl)porphine, N-methyl mesoporphyrin, diamidino-2phenylindole, 1-pyrenemethylamine hydrochloride, netropsin, hoeschst 33342, hoeschst 33258, hoeschst 8208, naphthalene diimide, and the like.

Suitable methods for preparing molecules having a portion that may be bound to a surface, such as an electrode, and a different portion comprising an intercalating group are disclosed in Higashi et al. Langmuir, 15, 111-115, 1999, which reference is incorporated herein.

In other embodiments, third portion 162 of second molecule 156 comprises a groove binder, which is configured to associate with a groove of a double-strand of DNA. The association may occur by non-covalent binding, such as by van der Waals forces and hydrogen bonding between the groove-binder and the double-strand of DNA. Exemplary groove binders comprise netropsin. The occurrence of groove binding may be determined by a modification of an electrical characteristic of a pair of electrodes. For example, the groove binding may reduce an electrical resistance or impedance between member electrodes of a pair of electrodes.

Referring to FIGS. 8d and 8e, sensor 100 may be used to determine the presence of one or more single stranded polynucleotides. For example, upon contacting the electrode pairs 144 of subset 103^{k} with a liquid comprising a second polynucleotide sequence 166 that is at least partially complementary to polynucleotide sequence 153 of first molecule 150, polynucleotide sequences 153 and 166 may form a duplex region, thereby forming a double stranded polynucleotide 168. (FIG. 8d). Second polynucleotide sequence 166 does not, however, form a duplex region with polynucleotide sequence 155 of molecule 152 because polynucleotide sequence 155 is different from polynucleotide sequence 153. If the electrode pairs 144 of subset 103^{k} are also contacted, e.g., simultaneously or sequentially, with a liquid comprising a second polynucleotide sequence 170 that is at least partially complementary to polynucleotide sequence 155 of first molecule 152, polynucleotide sequences 155 and 170 may form a duplex region, such as by forming a double stranded polynucleotide 172. (FIG. 8d).

As seen in FIG. 8e, intercalating groups of second molecules 156 associated with respective electrodes 106₁ and 106₂ may intercalate with double stranded polynucleotides 168 and 172 of electrodes 110₁ and 110₂, thereby forming an electrical connection between the electrodes of each pair. For example, electrons may travel between electrodes 110₁ and 106₁ along an electrical connection that comprises an intercalation complex 175 comprising double stranded polynucleotide 168 and intercalation group 162. The electrical path preferably comprises conductive oligomer 160. Similarly, electrons may travel between electrodes 110₂ and 106₂ along an electrical connection that comprises an intercalation complex 176 comprising double stranded polynucleotide 168 and intercalation group 162. By determining whether an electrical connection has been formed between the electrodes of an electrode pair, one may determine whether a particular target polynucleotide is present. For example, the formation of an electrical connection may be determined by measuring a resistance, an impedance, a capacitance, or a conductance of one or both electrodes of an electrode pair.

Array Preparation Apparatus

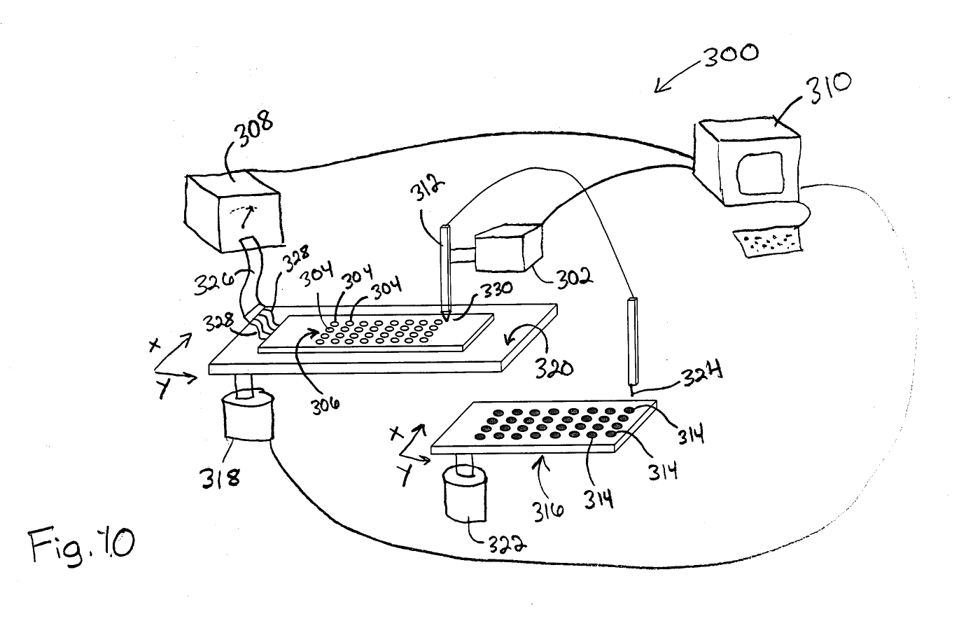

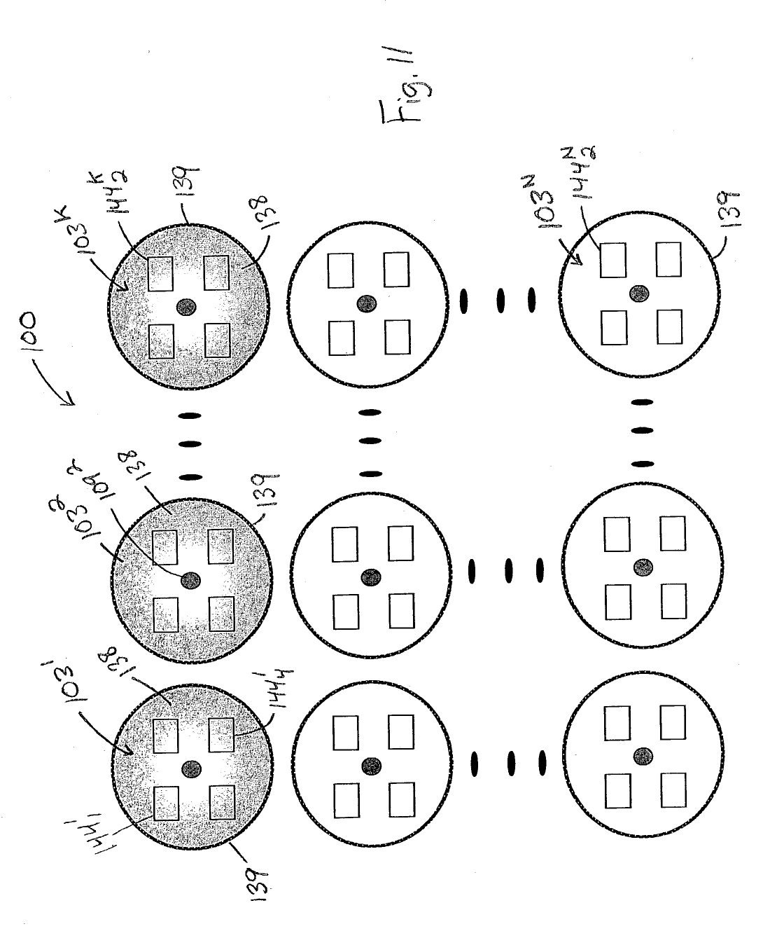

Referring to FIG. 10, an exemplary array preparation apparatus 300 for preparing an array of surface modified electrodes in accordance with the present invention comprises a liquid contacting device 302 configured to contact subsets of electrodes 304 of an array 306 of electrodes with at least one liquid comprising a molecule to be associated with one or more electrodes of array 306. An electrical potential modifying device 308 is configured to modify an electrical potential between selected electrodes of subsets 304 of array 306 and a reference electrode to thereby deprotect the selected electrodes allowing molecules present in the liquid to associate with the electrodes. A computer 310 controls liquid contacting device 302 and electrical potential modifying device 308.

Liquid contacting device preferably comprises at least one droplet preparation device 312 configured to apply one or more droplets of liquid to one or more subsets 304 of electrodes. Although, for clarity, only one droplet preparation device is shown, preferred embodiments of array preparation devices in accordance with the present invention include a plurality of droplet preparation devices 312, which may be configured to apply droplets of respective liquids comprising respective, different molecules to electrodes of the array.

Droplet preparation device 312 is in fluid communication with a plurality of reservoirs 314, each comprising a liquid comprising a molecule to be associated with an electrode of the array. Where multiple droplet devices 312 are used, each may be in fluid communication with a respective different reservoir 314. The reservoirs may be wells of a microtitre plate 316. Liquid contacting device 302 may comprise at least one introduction portion configured to receive liquid from respective reservoirs. For example, introduction portion may be configured to apply a vacuum to a tip 326, thereby drawing liquid therein.

Droplet preparation device 312 preferably comprises at least one ink jet nozzle configured to prepare droplets of liquid by thermally modifying a pressure of the liquid, or piezo-electrically modifying a pressure of the liquid, or ultrasonically modifying a pressure of the liquid. Alternatively, droplet preparation device 312 may comprise at least one capillary or micropipette configured to apply a droplet of liquid to one or more subsets 304. Examples of apparatus for applying liquids to substrates are disclosed in U.S. Pat. No. 6,479,301 to Balch et al., Fundamentals of Microfabrication, Second Edition, Marc J. Madou, CRC Press, Boca Raton, and U.S. Pat. No. 5,601,980 to Gordon et al. each of which is incorporated herein.

Array preparation apparatus also comprises a translation device 318 configured to translate array 306 and the one or more droplet preparation devices 312 with respect to one another. Preferably, the translation device translates a platform 320 supporting array 306 in at least two dimensions with respect to droplet preparation device 312 so that respective liquids may be applied to different subsets of the electrodes. Computer 310 may also control translation device. Apparatus 300 may comprise a second translation device configured to translate reservoirs 314 with respect to an introduction portion 324 of liquid contacting device 302. Each droplet preparation device 312 may comprise a introduction portion 324.

Referring to FIG. 11, array 100 is shown with liquid 138 contacting a plurality of subsets 103^{k} of electrodes. Liquid 138, may have been applied to each subset 103 in the form of one or more droplets of liquid, which are inhibited from spreading by liquid barriers 139. For example, liquid applied to respect subsets 103¹ and 103² does not establish electrical contact between electrodes of the respective subsets. It should be understood that the present invention does not require use of liquid barriers 139. For example, by limiting the amount of liquid applied to respective subsets of electrodes, spreading of liquid may be minimized with respect to the spacing between subsets.

Electrical potential modifying device 308 is electrically connected with the member electrodes (not shown) of each subset 304 of electrodes of array 306. (FIG. 10). For example, a connector 326, e.g., a ribbon cable, may connect potential modifying device 308 with platform 320. Array 306 is electrically connected with platform 320 via a plurality of leads, which are also in electrical connection with connector 326. Thus, electrical potential modifying device 308 may be configured to independently address each electrode of array 304, preferably by independently modifying an electrical potential difference between an addressed electrode and a reference electrode.

### EXAMPLE

The following example demonstrates the modification of electrodes of an array of electrodes by selective deprotection of electrodes of an array of electrodes.

(1) Protection of Bare Gold Electrodes

Bare gold electrodes were cleaned by contacting the electrodes with a solution of 70%H2SO4, 30% H2O2 for one minute to remove organic surface contaminants. Each electrode within the array was protected by forming a self-assembled monolayer of a thiol containing compound on the electrodes. The self-assembled monolayers were prepared by exposing the electrodes of the array to an aqueous solution of 1 mM mercaptohexanol for between 1 and 4 hours. Electrodes of the array were contacted with ethanol to remove any mercaptohexanol molecules not non-covalently bound to the electrodes.

(2) Deprotection of Target Electrode

Electrodes of the array were addressed to deprotect individual electrodes by removing the mercaptohexanol. An electrode to be deprotected was contacted with an aqueous solution comprising 0.1 M KOH for 100 seconds. A step voltage of −1.2 volts versus a reference electrode was applied to an electrode to be deprotected. In this example, the reference electrode was a Ag/Cl electrode, although other reference electrodes may be used. Upon application of the step voltage, the mercaptohexanol was reductively desorbed according to the reaction:

HO(CH₂)₆SAu (absorbed)+e−—>HO(CH₂)₆S—+Au

Only electrodes addressed by modifying the potential difference between the electrode and the reference electrode were deprotected.

(3) Attachment of Oligonucleotide Probe Sequence:

Upon deprotecting an electrode, electrodes of the array were exposed to a liquid comprising a high ionic strength buffered solution of a thiol-terminated oligonucleotide for between 1 and 4 hours. The thiol-terminated oligonucleotide reacted with the surfaces of electrodes that had been deprotected by desorbing the mercaptohexanol to form a self assembled layer of the thiol-terminated oligonucleotide. Mercaptohexanol bound to electrodes that had not been deprotected inhibited adsorption of the thiol-terminated oligonucleotide thereto.

The electrodes of the array were then re-exposed to a liquid comprising 1 mM mercaptohexanol for one hour and rinsed with water to prepare, at the surfaces of the deprotected electrodes, a stable phase capable of supporting hybridization to the thioterminated oligonucleotides.

The steps of deprotecting one or more electrodes and attaching a thiol-terminated oligonucleotide were repeated until a monolayer comprising a respective thiol-terminated oligonucleotide had been formed at the surface of each electrode within the array. The modified array may be exposed to a liquid comprising oligonucleotides at least partially complementary to the thiol-terminated electrodes of the electrode array. Hybridization between a thiol-terminated electrode and a partially complementary oligonucleotide may be determined by monitoring an electrical characteristic, such as a capacitance of each electrode within the array. Thus, the modified electrode array may be used to determine the presence of a plurality of polynucleotides.

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those of skill in the art. The specific embodiments described herein are offered by way of example only, and the invention is to be limited only by the terms of the appended claims, along with the full scope of equivalents to which such claims are entitled.

What is claimed is:

1. A method of modifying electrodes of an array of electrodes, the electrodes to be modified by binding at least one respective probe molecule thereto, wherein, prior to being modified, at least one respective, protective molecule overlays each of at least two electrodes to be modified such that the at least one respective, protective molecule inhibits probe molecules from binding to the at least two electrodes, the method comprising:
(a) dissociating the at least one respective protective molecule from at least one electrode overlaid by the at least one protective molecule; and
(b) contacting electrodes of each of a plurality of subsets of electrodes of the array of electrodes with a respective liquid, wherein each liquid comprises a respective, different probe molecule; and
wherein, at least one electrode is subjected to both the steps of (a) dissociating and (b) contacting and, for at least one electrode subjected to both the steps of (a) dissociating and (b) contacting, the respective, different probe molecule of the respective liquid binds to the electrode.

2. The method of claim 1, wherein at least 2 electrodes are subjected to both the steps of (a) dissociating and (b) contacting, and wherein at least two electrodes that are subjected to both the steps of (a) dissociating and (b) contacting, are members of respective, different subsets of electrodes.

3. The method of claim 2, wherein at least 25 electrodes that are subjected to both the steps of (a) dissociating and (b) contacting, are members of respective, different subsets of electrodes.

4. The method of claim 3, wherein at least 100 electrodes that are subjected to both the steps of (a) dissociating and (b) contacting, are members of respective, different subsets of electrodes.

5. The method of claim 1, wherein at least some subsets of the plurality of said subsets of electrodes comprise at least 2 member electrodes but fewer than 50 member electrodes.

6. The method of claim 1, wherein at least some subsets of the plurality of said subsets of electrodes comprise at least 5 member electrodes but fewer than 25 member electrodes.

7. The method of claim 1, wherein, for at least some subsets of the plurality of said subsets of electrodes, the step of (b) contacting is performed after the step of (a) dissociating.

8. The method of claim 1, wherein, for at least some subsets of the plurality of said subsets of electrodes, the step of (b) contacting is performed after initiating the step of (a) dissociating.

9. The method of claim 1, wherein, for at least some subsets of the plurality of said subsets of electrodes, the step of (a) dissociating is performed while the subsets of electrodes are in contact with the respective liquids of the step of (b) contacting.

10. The method of claim 1, wherein the step of (b) contacting comprises:
contacting each subset of a first portion of the plurality of said subsets with the respective liquid; and
while the subsets of the first portion of subsets remain in contact with the respective liquids, contacting each subset of a second, different portion of the plurality of said subsets with the respective liquid.

11. The method of claim 10, wherein, while performing the step of (b) contacting, at least 25 of said subsets of electrodes are in simultaneous contact with the respective liquid comprising a respective, different molecule.

12. The method of claim 11, wherein, while performing the step of (b) contacting, at least 100 of said subsets of electrodes in simultaneous contact with the respective liquid comprising a respective, different molecule.

13. The method of claim 1, wherein the step of (b) contacting comprises simultaneously contacting at least some subsets of the plurality of said subsets of electrodes with the respective liquid.

14. The method of claim 1, wherein the respective liquids comprise at least two different liquids.

15. The method of claim 1, wherein, for each electrode of a plurality of the electrodes, the step of (a) dissociating comprises modifying an electrical potential of the electrode, whereby the at least one respective, protective molecule dissociates from the electrode.

16. The method of claim 1, wherein, for each electrode of a plurality of the electrodes, the step of (a) dissociating comprises modifying an electrical potential difference between the electrode and a reference electrode, whereby the at least one respective, protective molecule dissociates from the electrode.

17. The method of claim 16, wherein, for each of at least two subsets of the plurality of said subsets of electrodes, the step of (b) contacting further comprises contacting a reference electrode with the respective liquid, thereby electrically contacting the electrodes of the subset of electrodes and the reference electrode.

18. The method of claim 16, wherein, for each of at least two subsets of the plurality of said subsets of electrodes, the step of (b) contacting further comprises contacting a respective, different reference electrode with the respective liquid, thereby electrically contacting the electrodes of the subset of electrodes and the respective, different reference electrode.

19. The method of claim 18, wherein, for each of at least two subsets of the plurality of said subsets of electrodes, the liquid used in the step of (b) contacting does not electrically connect the electrodes of the subset with the respective reference electrodes of other subsets of electrodes.

20. The method of claim 18, wherein, for each of at least two subsets of the plurality of said subsets of electrodes and the respective, different reference electrode thereof, the step of (b) contacting comprises applying at least one droplet of liquid to the subset of electrodes and reference electrode, each droplet of liquid comprising at least one of the respective, different probe molecules.

21. The method of claim 1, wherein, for each of at least two subsets of the plurality of said subsets of electrodes, the step of (b) contacting comprises applying at least one droplet of liquid to the subset of electrodes, each droplet of liquid comprising at least one of the respective, different probe molecules.

22. The method of claim 1, further comprising:
repeating the steps of (a) dissociating and (b) contacting until a respective probe molecule is bound to each of at least 50 electrodes of the array.

23. The method of claim 22, further comprising:
repeating the steps of (a) dissociating and (b) contacting until a respective probe molecule is bound to each of at least 500 electrodes of the array.

24. The method of claim 1, further comprising:
repeating the steps of (a) dissociating and (b) contacting until a respective probe molecule is bound to every electrode of the array.

25. The method of claim 1, further comprising:
prior to performing the steps of (a) dissociating and (b) contacting, overlaying a plurality of the electrodes with at least one protective molecule by contacting the electrodes with a liquid comprising the at least one protective molecule, wherein at least one respective protective molecule binds to electrodes of the array.

26. The method of claim 25, wherein the at least one protective molecule comprises at least one of an alkylsiloxane, an alkylthiolate, and a fatty acid.

27. The method of claim 26, wherein the alkylthiolate comprises an alkanethiol having from 1 to 22 carbon atoms.

28. The method of claim 23, wherein, for each electrode of a plurality of electrodes, the at least one respective, protective molecule binds to the electrode by a sulfur group.

29. The method of claim 1, wherein the at least one of the respective, protective molecules comprises at least one of an alkylsiloxane, an alkylthiolate, and a fatty acid.

30. The method of claim 29, wherein the alkylthiolate comprises an alkanethiol having from 1 to 22 carbon atoms.

31. The method of claim 29, wherein, for each electrode of a plurality of electrodes, the at least one respective, protective molecule is bound to the electrode by a sulfur group.

32. The method of claim 1, wherein the probe molecules each comprise a polynucleotide.

33. The method of claim 32, wherein the polynucleotides of probe molecules bound to different electrodes have different sequences from one another.

34. The method of claim 32, wherein the probe molecules comprise a binding portion that binds the electrodes, the binding portion comprising sulfur.

35. The method of claim 1, wherein the array of electrodes comprises a plurality of electrode pairs, each electrode pair comprising first and second electrodes that are spaced apart by less than 1000 Angstroms, and wherein:
for at least one electrode pair of the plurality of said electrode pairs, the step of (a) dissociating comprises dissociating the at least one respective, protective molecule from only the first electrode of the electrode pair; and
for at least one electrode pair of the plurality of said electrode pairs, the step of (b) contacting comprises contacting both electrodes of the electrode pair with the same respective liquid comprising the same respective, different problem molecule; and
wherein, for at least one electrode pair of the plurality of said electrode pairs, the electrode pair is subjected to the step of (b) contacting and the first electrode only of the electrode pair is also subjected to the step of (a) dissociating, and wherein the respective, different probe molecule of the respective liquid binds only to the first electrode.

36. The method of claim 35, wherein the first and second electrodes of the electrode pairs of the array are spaced apart by less than 500 Angstroms.

37. The method of claim 35, wherein:
for each electrode pair of at least two electrode pairs of the plurality of said electrode pairs, the step of (a) dissociating comprises dissociating the at least one respective, protective molecule from only the first electrode of the electrode pair;
for each electrode pair of at least two electrode pairs of the plurality of electrode pairs, the electrode pairs belong to different subsets of the plurality of subsets of electrodes and the step of (b) contacting comprises contacting the at least two electrode pairs with respective liquids comprising respective, different probe molecules; and
wherein for each electrode pair of at least two electrode pairs contacted with respective liquids comprising respective, different probe molecules, only the first electrode of the electrode pair is also subjected to the step of (a) dissociating, and wherein the respective, different probe molecule of the respective liquid binds only to the first electrode.

38. The method of claim 35, wherein for at least one electrode pair having had the first electrode subjected to both the steps of (a) dissociating and (b) contacting, the method further comprises:
dissociating the at least one protective molecule from the second electrode of the electrode pair; and
contacting both electrodes of the electrode pair with a liquid comprising a probe molecule to be bound to the second electrode of the electrode pair, wherein the probe molecule to be bound to the second electrode is different from the probe molecule bound to the first electrode; and
wherein the probe molecule to be bound to the second electrode of electrode pair binds to the second electrode.

39. The method of claim 38, wherein, the probe molecule bound to one of the first and second electrodes comprises a polynucleotide.

40. The method of claim 35, wherein, for each electrode pair of at least two of the electrode pairs, the probe molecule bound to the other electrode comprises an intercalating group and wherein, upon contacting the electrode pair with a liquid comprising a target polynucleotide at least partially complementary to the first polynucleotide of the probe molecule bound the first electrode, the first and target polynucleotides will form a duplex region and the intercalating group will intercalate with the duplex region.

41. The method of claim 35, wherein, for each electrode pair of at least two of the electrode pairs, the probe molecule bound to the other electrode comprises an intercalating group and wherein, upon contacting the electrode pair with a liquid comprising a target polynucleotide at least partially complementary to the first polynucleotide of the probe molecule bound to the first electrode an electrical resistance between the first and second electrodes will be reduced.

42. The method of claim 1, further comprising:
for at least one electrode to which a respective, different probe molecule is bound, contacting the electrode with a liquid comprising a second protective molecule, wherein the second protective molecule also binds to the electrode.

43. A method of modifying electrodes of an array of electrode pairs, each electrode pair comprising a first and second electrode, the first and second electrodes of the electrode pairs to be modified by binding at least one respective probe molecule thereto, wherein, prior to being modified, at least one respective, protective molecule overlays each of the first and second electrodes of at least one electrode pair such that the at least one respective, protective molecule inhibits probe molecules from binding to the first and second electrodes, the method comprising:
(a) dissociating the at least one protective molecule from the first electrode of at least one electrode pair without dissociating the at least one protective molecule from the second electrode of the at least one electrode pair, the first and second electrodes of the at least one electrode pair being spaced apart by less than 1000 Angstroms; and
(b) contacting the first and second electrode of at least one electrode pair of the array of electrode pairs with a liquid comprising a first probe molecule; and
wherein, for at least one first electrode of at least one electrode pair subjected to the step of (b) contacting, the first electrode is also subjected to the step of (a) dissociating, wherein the first probe molecule of the liquid binds to the first electrode.

44. The method of claim 43, further comprising:
for at least one electrode pair comprising a first electrode to which the first probe molecule was bound, (c) dissociating the at least one protective molecule from the second electrode of the at least one electrode pair;
(d) contacting electrodes of each of a second plurality of electrode pairs of the array of electrode pairs with a liquid comprising a second probe molecule to be bound to a second electrode of at least one electrode pair; and
wherein, at least one second electrode is subjected to both the steps of (c) dissociating and (d) contacting and for, each second electrode subjected to both the steps of (c) dissociating and (d) contacting, the second probe molecule of the liquid binds to the second electrode.

45. The method of claim 43, wherein the first probe molecule comprises a polynucleotide.

46. The method of claim 45, wherein the polynucleotide of the first probe molecule comprises a phosphorothiolated polynulceotide.

47. The method of claim 44, wherein the second probe molecule comprises an intercalating group configured to intercalate with double stranded polynucleotides.

48. A method of modifying electrodes of an array of electrodes, electrodes of the array to be modified by binding at least one respective probe molecule thereto, wherein, prior to being modified, at least one respective protective molecule overlays each of at least two electrodes to be modified such that the at least one respective, protective molecule inhibits probe molecules from binding to electrodes of the at least two electrodes, the method comprising:
(a) contacting a plurality of electrodes of the array of electrodes with a liquid comprising a probe molecule;
(b) dissociating the at least one protective molecule from at least one of the electrodes in contact with the liquid comprising the probe molecule, wherein, for each electrode in contact with the liquid and subjected to the step of (b) dissociating, the probe molecule of the liquid binds to the electrode.

49. The method of claim 48, wherein the step of dissociating is performed without first removing the liquid used in the step of (a) contacting.

50. The method of claim 48, wherein, for at least one electrode, the step of (b) dissociating comprises modifying an electrical potential of the at least one electrode.

51. The method of claim 48, wherein, for at least one electrode, the step of (b) dissociating comprises modifying an electrical potential difference between the at least one electrode and a reference electrode.

52. The method of claim 48, further comprising:
(c) contacting a plurality of electrodes of the array of electrodes with a liquid comprising a different, probe molecule; and
(d) dissociating the at least one protective molecule from at least one electrode in contact with the liquid used in the step of (c) contacting, wherein, the different, probe molecule of the liquid binds to the at least one electrode.

53. The method of claim 52, wherein, for at least one electrode, the step of (d) dissociating comprises modifying an electrical potential of the at least one electrode, whereby the at least one molecule dissociates from the at least one electrode.

54. The method of claim 52, wherein, for at least one electrode, the step of (d) dissociating comprises modifying an electrical potential difference between the at least one electrode and a reference electrode, whereby the at least one molecule dissociates from the at least one electrode.

55. The method of claim 52, further comprising:
repeating the steps of (c) dissociating and (d) contacting until a respective probe molecule is bound to each of at least 50 electrodes of the array.

56. The method of claim 52, further comprising:
repeating the steps of (c) dissociating and (d) contacting until a respective probe molecule is bound to at least 500 electrodes of the array.

57. The method of claim 52, further comprising:
repeating the steps of (c) dissociating and (d) contacting until a respective probe molecule is bound to every electrode of the array.

58. The method of claim 48, further comprising:
prior to performing the steps of (a) contacting and (b) dissociating, overlaying each of a plurality of the electrodes with at least one protective molecule by contacting the electrodes with a liquid comprising the at least one protective molecule, wherein at least respective one protective molecule binds to electrodes of the array.

59. The method of claim 58, wherein the at least one of the respective, protective molecules comprises at least one of an alkylsiloxane, an alkylthiolate, and a fatty acid.

60. The method of claim 59, wherein the alkylthiolate comprises an alkane thiol having from 1 to 22 carbon atoms.

61. The method of claim 58, wherein, for each electrode of a plurality of electrodes, the at least one respective, protective molecule binds to the electrode by a sulfur group.

62. The method of claim 48, wherein the at least one protective molecule comprises at least one of an alkylsiloxane, an alkylthiolate, and a fatty acid.

63. The method of claim 62, wherein the alkylthiolate comprises an alkane thiol having from 1 to 22 carbon atoms.

64. The method of claim 48, wherein, for each electrode of a plurality of electrodes, the at least one protective molecule is bound to the electrode by a sulfur group.

65. The method of claim 48, wherein the probe molecules each comprise a polynucleotide.

66. The method of claim 65, wherein the polynucleotides of each of a plurality of the probe molecules have different sequences.

67. The method of claim 65, wherein the probe molecules comprise a binding portion that binds the electrodes, the binding portion comprising at least one sulfur atom.

68. The method of claim 48, wherein the array of electrodes comprises a plurality of electrode pairs, each electrode pair comprising first and second electrodes that are spaced apart by less than 1000 Angstroms, and wherein:
for at least one electrode pair of the plurality of said electrode pairs, the step of (a) dissociating comprises dissociating the at least one respective, protective molecule from only the first electrode of the electrode pair; and
for at least one electrode pair of the plurality of said electrode pairs, the step of (b) contacting comprises contacting both electrodes of the electrode pair with the same respective liquid comprising the same respective, different problem molecule; and
wherein, for at least one electrode pair of the plurality of said electrode pairs, the electrode pair is subjected to the step of (b) contacting and the first electrode only of the electrode pair is also subjected to the step of (a) dissociating, and wherein the respective, different probe molecule of the respective liquid binds only to the first electrode.

69. The method of claim 68, wherein,
for each electrode pair of at least two electrode pairs of the plurality of said electrode pairs, the step of (a) dissociating comprises dissociating the at least one respective, protective molecule from only the first electrode of the electrode pair;
for each electrode pair of at least two electrode pairs of the plurality of electrode pairs, the electrode pairs belong to different subsets of the plurality of subsets of electrodes and the step of (b) contacting comprises contacting the at least two electrode pairs with respective liquids comprising a respective, different probe molecules; and
wherein for each electrode pair of at least two electrode pairs contacted with respective liquids comprising respective, different probe molecules, only the first electrode of the electrode pair is also subjected to the step of (a) dissociating, and wherein the respective, different probe molecule of the respective liquid binds only to the first electrode.

70. The method of claim 68, wherein for at least one each electrode pair having had the first electrode subjected to both the steps of (b) dissociating and (c) contacting, the method further comprises:
dissociating the at least one protective molecule from the second electrode of the electrode pair;
contacting both electrodes of the electrode pair with a liquid comprising a probe molecule to be bound to the second electrode of the electrode pair, wherein the probe molecule to be bound to the second electrode is different from the probe molecule bound to the first electrode; and
wherein the probe molecule to be bound to the second electrode of electrode pair binds to the second electrode.

71. The method of claim 70, wherein, for each electrode pair of a plurality of electrode pairs, the probe molecule bound to one of the first and second electrodes comprises a first polynucleotide.

72. The method of claim 71, wherein, for each electrode pair of a plurality of electrode pairs, the probe molecule bound to the other electrode comprises an intercalating group and wherein, upon contacting the electrode pair with a liquid comprising a target polynucleotide at least partially complementary to the first polynucleotide of the probe molecule bound to the first electrode, the first and target polynucleotides form a duplex region and the intercalating group intercalates with the duplex region polynucleotides.

73. A method of modifying electrodes of an array of electrodes, the electrodes to be modified by binding at least one respective probe molecule thereto, the method comprising:
(a) addressing at least one electrode of the array of electrodes with a dissociation potential;
(b) contacting electrodes of the array of electrodes with a liquid comprising a probe molecule;
(c) contacting electrodes of the array of electrodes with a liquid comprising a protective molecule; and
wherein at least a first electrode subjected to the step of (a) addressing is (i) subjected to the step of (b) contacting while not concurrently being subjected to the step of (a) addressing and (ii) subjected to the step of (c) contacting while not concurrently being subjected to the step of (a) addressing, and wherein at least one probe molecule and at least one protective molecule bind to the first electrode.

74. The method of claim 60, further comprising:
repeatedly:
(d) addressing at least one different electrode with a dissociation potential;
(e) contacting electrodes of the array with a liquid comprising a different probe molecule;
(f) contacting electrodes of the array with a liquid comprising a protective molecule; and
wherein at least a second electrode subjected the step of (d) addressing is (1) subjected to a step of (e) contacting while not concurrently being subjected to a step of (d) addressing and (2) subjected to a step of (f) contacting while not concurrently being subjected to a step of (d) addressing, and wherein at least one different probe molecule and at least one protective molecule bind to the second electrode.

75. The method of claim 73, further comprising:
(g) addressing at least one electrode of the array of electrodes with a dissociation potential, wherein at least one electrode that was subjected to the step of (a) addressing and was (1) subjected to the step of (b) contacting while not concurrently being subjected to the step of (a) addressing and (2) subjected to the step of (c) contacting while not concurrently being subjected to the step of (a) addressing is not subjected to the step of (g) addressing;
(h) contacting electrodes of the array of electrodes with a liquid comprising a different probe molecule;
(i) contacting electrodes of the array of electrodes with a liquid comprising a protective molecule; and
wherein at least a second electrode subjected to the step of (g) addressing is (1) subjected to the step of (h) contacting while not concurrently being subjected to the step of (g) addressing and (2) subjected to the step of (i) contacting while not concurrently being subjected to the step of (g) addressing, and wherein at least one probe molecule and at least one protective molecule bind to the second electrode.

76. The method of claim 73, wherein the step of (a) addressing comprises modifying an electrical potential of the at least one electrode.

77. The method of claim 73, wherein the step of (a) addressing comprises modifying an electrical potential difference between the at least one electrode and a reference electrode.

78. The method of claim 73, wherein the step of (c) contacting is performed after the step of (b) contacting.

79. The method of claim 73, wherein at least one of the steps of (b) contacting and (c) contacting are performed after the step of (a) addressing.

80. The method of claim 73, further comprising:
prior to the steps of (a) addressing, (b) contacting, and (c) contacting, overlaying a plurality of the electrodes with at least one respective, protective molecule by contacting the electrodes with a liquid comprising the at least one respective, protective molecule, wherein at least one respective, protective molecule binds to electrodes of the array.

81. The method of claim 80, wherein the step of (a) addressing dissociates the at least one protective molecule from the at least one electrode.

82. The method of claim 80, wherein the at least one protective molecule comprises at least one of an alkylsiloxane, an alkylthiolate, and a fatty acid.

83. The method of claim 82, wherein the alkylthiolate comprises an alkane thiol having from 1 to 22 carbon atoms.

84. The method of claim 80, wherein, for each electrode of a plurality of electrodes, the at least one protective molecule binds to the electrode by a sulfur group.

85. The method of claim 73, wherein the at least one protective molecule comprises at least one of an alkylsiloxane, an alkanethiolate, and a fatty acid.

86. The method of claim 85, wherein the alkylethiolate comprises an alkane thiol having from 1 to 22 carbon atoms.

87. The method of claim 85, wherein, for each electrode of a plurality of electrodes, the at least one protective molecule is bound to the electrode by a sulfur group.

88. The method of claim 74, wherein the probe molecules each comprise a polynucleotide

89. The method of claim 88, wherein the polyntucleotides of different probe molecules have different sequences.

90. The method of claim 88, wherein the probe molecules comprise a binding portion that binds the electrodes, the binding portion comprising sulfur.

91. A method of forming an electrical connection between a first electrode and a second electrode of an electrode pair:
binding a first molecule to the first electrode, the first molecule comprising a first single stranded polynucleotide;
binding a second molecule to the second electrode, the second molecule comprising an intercalating group configured to intercalate with double stranded polynucleotides; and
contacting the electrode pair with a second single stranded polynucleotide at least partially complementary to the first polynucleotide, wherein the first and second polynucleotides form a duplex region and the intercalating group intercalates with the duplex region thereby forming the electrical connection between the first and second electrodes.

92. The method of claim 91, wherein binding the first molecule to the first electrode comprises binding a sulfur group of the first molecule to the first electrode.

93. The method of claim 92, wherein sulfur group comprises a phosphorothioate group.

94. The method of claim 91, wherein the second molecule comprises a conductive oligomer disposed intermediate the intercalating group and a second portion of the second molecule that is associated with the second electrode.

95. The method of claim 91, wherein the second molecule is free of polynucleotides.

96. The method of claim 91, wherein binding the second molecule to the second electrode comprises binding a sulfur group of the second molecule to the second electrode.

97. The method of claim 91, wherein the intercalating group comprises at least one of (i) ethidium bromide or acridine and (ii) a derivative of ethidium bromide or a derivative or acridine.

98. The method of claim 91, comprising:
prior to the step of binding the first molecule to the first electrode, overlaying at least one protective molecule upon the first electrode, whereby the at least one protective molecule inhibits association of the first and second molecules with the first electrode;
wherein the step of binding the first molecule to the first electrode comprises:
contacting the first and second electrodes with a liquid comprising the first molecule; and
modifying an electrical potential difference between the first electrode and a reference electrode to thereby deprotect the first electrode, whereupon the first molecule binds to the first electrode.

99. The method of claim 98, comprising:
prior to the step of binding the second molecule to the second electrode, overlaying at least one protective molecule upon the second electrode, whereby the at least one protective molecule inhibits association of the first and second molecules with the second electrode;
wherein the step of binding the second molecule to the second electrode comprises:
contacting the first and second electrodes with a liquid comprising the first molecule; and
modifying an electrical potential difference between the second electrode and a reference electrode to thereby deprotect the second electrode, whereupon the second molecule binds to the second electrode.

100. The method of claim 91, wherein the method further comprises forming a respective electrical connection between a first and a second electrode of each of a plurality of electrode pairs, for each electrode, the method comprising:
binding a first molecule to the first electrode, the first molecule comprising a first polynucleotide;
binding a second molecule to the second electrode, the second molecule comprising an intercalating group configured to intercalate with double stranded polynucleotide compounds; and
contacting the first and second molecules with a second polynucleotide at least partially complementary to the first polynucleotide, wherein the first and second polynucleotides form a duplex region and the intercalating group intercalates with the duplex region thereby forming the electrical connection between the first and second electrodes.

101. The method of claim 100, comprising binding first molecules comprising respective, different first polynucleotides to the first electrodes of respective, different electrode pairs, whereby the first polynucleotides bound to different first electrodes will selectively form duplex regions with different, second polynucleotides.

102. The method of claim 100, wherein, for each electrode pair, the method comprises:
prior to the step of binding the first molecule to the first electrode, overlaying at least one protective molecule upon the first electrode, whereby the at least one protective molecule inhibits binding of the first and second molecules with the first electrode;
wherein the step of binding the first molecule to the first electrode comprises:
contacting the first and second electrodes with a liquid comprising the first molecule; and
modifying an electrical potential difference between the first electrode and a reference electrode to thereby deprotect the first electrode whereupon the first molecule binds to the first electrode.

103. The method of claim 102, wherein, for each electrode pair, the method comprises:
prior to the step of binding the second molecule to the second electrode, overlaying at least one protective molecule upon the second electrode, whereby the at least one protective molecule inhibits binding of the first and second molecules with the second electrode;
wherein the step of binding the second molecule with the second electrode comprises:
contacting the first and second electrodes with a liquid comprising the second molecule; and
modifying an electrical potential difference between the second electrode and a reference electrode to thereby deprotect the second electrode whereupon the second molecule binds to the second electrode.

104. The method of claim 100, wherein, for each electrode pair, the step of binding a first molecule to the first electrode comprises:
contacting at least two subsets of the electrode pairs with a respective liquid, wherein each liquid comprises a respective, different first molecule; and
for each of at least two subsets of electrode pairs, modifying an electrical potential difference between the first electrode of at least one of the electrode pairs and a reference electrode, whereby the respective first molecule binds with the first electrode.

105. The method of claim 104, comprising:
contacting at least two subsets of the electrode pairs with a respective liquid, wherein each liquid comprises a respective, different compound; and
for each of at least two subsets of electrode pairs, modifying an electrical potential difference between the first electrode of at least one of the electrode pairs and a reference electrode, whereby the respective first molecule binds to the first electrode.

106. The method of claim 105, comprising:
repeating the steps of contacting at least two subsets of electrode pairs and modifying an electrical potential difference between the first electrode of at least one electrode pair of each subset until each of the first electrodes has been bound with a respective first molecule.

107. The method of claim 87, wherein, for each electrode pair, the step of binding a second molecule to the second electrode comprises:
contacting a number N subsets of the electrode pairs with a respective liquid, wherein each liquid comprises a respective, different second molecule and N is an integer greater than 1 and less than N^{a}; and
for each subset of the N subsets of electrode pairs, modifying an electrical potential difference between the second electrode of at least one of the electrode pairs and a reference electrode, whereby the respective second molecule binds to the second electrode.

108. The method of claim 107, comprising:
contacting a number N′ subsets of the electrode pairs with a respective liquid, wherein each liquid comprises a respective, different compound and N′ is an integer greater than 1 and less than N^{a}; and
for each subset of the N′ subsets of electrode pairs, modifying an electrical potential difference between the second electrode of at least one of the electrode pairs and a reference electrode, whereby the respective second molecule binds to the second electrode.

109. The method of claim 108, comprising:
repeating the steps of contacting subsets of electrode pairs and modifying an electrical potential difference between the second electrode of at least one electrode pair of each subset until each of the second electrodes has been bound with a respective second molecule.

110. A method of preparing a sensor, the method comprising:
binding a first molecule to a first electrode, the first molecule comprising a first single stranded polynucleotide;
binding a second molecule to a second electrode, the second molecule comprising an intercalating group configured to intercalate with double stranded polynucleotides;
wherein, if the first electrode pair is contacted with a liquid comprising a second single stranded polynucleotide sequence at least partially complementary to the first polynucleotide sequence, the first and second polynucleotide sequences will form a duplex region and the intercalating group will intercalate with the duplex region thereby modifying an electrical characteristic of the first and second electrodes, whereby the presence of the at least partially complementary polynucleotide may be determined.

111. The method of claim 110, wherein binding the first molecule with the first electrode comprises binding a sulfur group of the first molecule with the first electrode.

112. The method of claim 110, wherein the sulfur group comprises a phosphorothioate group.

113. The method of claim 110, wherein the second molecule comprises a conductive oligomer disposed intermediate the intercalating group and a portion of the second molecule that is bound to the second electrode.

114. The method of claim 113, wherein the conductive oligomer comprises at least one of a saccharide and an aromatic group.

115. The method of claim 113, wherein the conductive oligomer is free of polynucleotides.

116. The method of claim 114, wherein the portion of the second molecule that is bound to the second electrode comprises sulfur.

117. The method of claim 114, wherein the intercalating group comprises at least one of (i) ethidium bromide or acridine and (ii) a derivative of ethidium bromide or a derivative of acridine.

118. The method of claim 113, comprising:
prior to the step of binding the first molecule to the first electrode, overlaying at least one protective molecule upon the first electrode, whereby the at least one protective molecule inhibits binding of the first and second molecules to the first electrode;
wherein the step of binding the first molecule to the first electrode comprises:
contacting the first and second electrodes to with a liquid comprising the first molecule; and
modifying an electrical potential difference between the first electrode and a reference electrode to thereby deprotect the first electrode, whereupon the first molecule binds to the first electrode.

119. The method of claim 118, comprising:
prior to the step of binding the second molecule with the second electrode, overlaying at least one protective molecule upon the second electrode, whereby the at least one protective molecule inhibits binding of the first and second molecules to the second electrode;
wherein the step of binding the second molecule to the second electrode comprises:
contacting the first and second electrodes with a liquid comprising the second molecule; and
modifying an electrical potential difference between the second electrode and a reference electrode to thereby deprotect the second electrode whereupon the second molecule binds with the second electrode.

120. The method of claim 110, wherein the substrate comprises an electrode pair array comprising a number N^{a} electrode pairs, each electrode pair comprising a first and second electrode pair and wherein, for each electrode pair, the method comprises:
binding a first molecule to the first electrode, the first molecule comprising a first polynucleotide;
binding a second molecule to a second electrode, the second molecule comprising an intercalating group configured to intercalate with double stranded polynucleotide compounds; and
wherein, if the first electrode pair is contacted with a liquid comprising a second polynucleotide sequence at least partially complementary to the first polynucleotide sequence, the first and second polynucleotide sequences will form a duplex region and the intercalating group will intercalate with the duplex region of the first and complementary polynucleotides thereby modifying an electrical characteristic of the first and second electrodes whereby the presence of the at least partially complementary polynucleotide may be determined.

121. The method of claim 120, comprising binding first molecules comprising respective, different first polynucleotides to the first electrodes of respective, different electrode pairs, whereby the first polynucleotides bound to different first electrodes will selectively form duplex regions with different second polynucleotides.

122. The method of claim 120, wherein, for each electrode pair, the method comprises:
prior to the step of binding the first molecule to the first electrode, binding at least one protective compound to the first electrode, whereby the at least one protective compound inhibits binding of the first and second molecules to the first electrode;
wherein the step of binding the first molecule to the first electrode comprises:
contacting the first and second electrodes with a liquid comprising the first molecule; and
modifying an electrical potential difference between the first electrode and a reference electrode to thereby deprotect the first electrode whereupon the first molecule binds to the first electrode.

123. The method of claim 120 wherein, for each electrode pair, the method comprises:
prior to the step of binding the second molecule to the second electrode, binding at least one protective compound with the second electrode, whereby the at least one protective compound inhibits binding of the first and second molecules to the second electrode;
wherein the step of binding the second molecule to the second electrode comprises:
contacting the first and second electrodes with a liquid comprising the second molecule; and
modifying an electrical potential difference between the second electrode and a reference electrode to thereby deprotect the second electrode whereupon the second molecule binds to the first electrode.

124. The method of claim 120, wherein, for each electrode pair, the step of binding a first molecule with the first electrode comprises:
contacting a number N subsets of the electrode pairs with a respective liquid, wherein each liquid comprises a respective, different first molecule and N is an integer greater than 1 and less than N^{a}; and
for each subset of the N subsets of electrode pairs, modifying an electrical potential between the first electrode of at least one of the electrode pairs and a reference electrode, whereby the respective first molecule binds to the first electrode.

125. The method of claim 124, comprising:
contacting a number N′ subsets of the electrode pairs with a respective liquid, wherein each liquid comprises a respective, different compound and N′ is an integer greater than 1 and less than N^{a}; and
for each subset of the N′ subsets of electrode pairs, modifying an electrical potential between the first electrode of at least one of the electrode pairs and a reference electrode, whereby the respective first molecule binds to the first electrode.

126. The method of claim 125, comprising:
repeating the steps of contacting subsets of electrode pairs and modifying an electrical potential until each of the first electrodes has been bound to a respective first molecule.

127. The method of claim 120, wherein, for each of the N subsets of electrode pairs, contacting the subset with a respective liquid comprises applying at least one aliquot of the respective liquid to the subset.

128. The method of claim 127, wherein the electrode pairs of each subset of electrode pairs are isolated from aliquots of liquid applied to other subsets of electrode pairs.

129. A method of forming an electrical connection between a first electrode and a second electrode of an electrode pair, the electrode pair comprising the first and second electrodes, wherein a surface of the first electrode is bound with a first molecule, the first molecule comprising a first single stranded polynucleotide and a surface of the second electrode is bound with a second molecule, the second molecule comprising an intercalating group configured to intercalate with double stranded polynucleotides, comprising:
contacting the first and second molecules with a second single stranded polynucleotide at least partially complementary to the first polynucleotide, wherein the first and second polynucleotides form a duplex region and the intercalating group intercalates with the first and second polynucleotides thereby forming the electrical connection between the first and second electrodes.

130. The method of claim 129, further comprising determining an electrical characteristic of the first and second electrodes whereby the presence of the second polynucleotide may be determined.

131. The method of claim 130, wherein the electrical characteristic is a conductance, a resistance, an impedance, or a capacitance.

132. The method of claim 129, wherein the first molecule comprises a sulfur group bound to the first electrode.

133. The method of claim 132, wherein the sulfur group comprises a phosphorothioate group.

134. The method of claim 132, wherein the second molecule comprises a conductive oligomer disposed intermediate the intercalating group and a portion of the second molecule that is bound to second electrode.

135. The method of claim 134, wherein the conductive oligomer comprises at least one of a saccharide and an aromatic group.

136. The method of claim 134, wherein the conductive oligomer is essentially free of polynucleotides.

137. The method of claim 129, wherein the intercalating group comprises at least one of ethidium bromide, acridine, and derivatives thereof.

138. An apparatus for preparing an array of modified surfaces, comprising:
a device configured to at least:
contact electrodes of each of a number N subsets of electrodes an array of electrodes with a respective liquid, wherein each liquid comprises a respective, different compound and N is an integer greater than 1; and
for each subset of the N subsets of electrodes, modify an electrical potential between at least a first electrode of the subset of electrodes and a reference electrode, whereby the respective compound of the fluid contacting the first electrode binds to the first electrode.

139. The apparatus of claim 138, wherein the device is configured to:
contact surfaces of each of a number N′ subsets of the electrodes of the array of electrodes with a respective liquid, wherein each liquid comprises a respective, different compound and N′ is an integer greater than 1;
for each subset of the N′ subsets of electrodes, modify an electrical potential between at least a second electrode and a reference electrode, whereby the respective compound binds to the second electrode.

140. The apparatus of claim 138, wherein the device is configured to:
repeatedly contact subsets of surfaces of the array of surfaces with a respective liquid, each liquid comprising a respective, different compound; and
modify an electrical potential between at least one electrode of the subset of electrodes and a reference electrode until a respective, different compound has been bound with each electrode of the array of electrodes.

141. The apparatus of claim 138, wherein the device comprises:
a plurality of droplet preparation devices, wherein each droplet preparation device is in fluid communication with a respective reservoir comprising a respective one of the different compounds; and
a droplet delivery device configured to deliver droplets prepared by the droplet preparation devices to predetermined subsets of the N subsets of electrodes to thereby contact the predetermined subsets with respective liquid.

142. The apparatus of claim 141, wherein the droplet preparation devices each comprise a capillary configured to prepare a droplet of fluid.

143. The apparatus of claim 141, wherein the droplet preparation devices are configured to prepare droplets by at least one of thermally modifying a pressure of the liquid, piezo-electrically modifying a pressure of the liquid, and ultrasonically modifying a pressure of the liquid.

144. The apparatus of claim 138, wherein the device is configured to:
bind at least one respective, protective molecule to the electrodes of the array, whereby the at least one respective, protective compound inhibits association of the respective, different compounds with electrodes.

145. A sensor, comprising:
a substrate comprising a first electrode pair comprising first and second electrodes;
a first molecule bound with the first electrode, the first molecule comprising a first polynucleotide;
a second molecule bound with the second electrode, the second molecule comprising a group configured to intercalate with double stranded polynucleotide compounds; and
wherein, upon contacting the first electrode pair with a liquid comprising a second polynucleotide sequence at least partially complementary to the first polynucleotide sequence, the first and second polynucleotide sequences form a duplex region and the intercalating portion intercalates with the duplex region, thereby modifying an electrical characteristic of the first and second electrodes whereby the presence of the at least partially second polynucleotide may be determined.

146. The sensor of claim 145, wherein the modified electrical characteristic comprises at least one of a conductance, a resistance, an impedance, and a capacitance.

147. The sensor of claim 145, wherein the substrate comprises a number N^{a} electrode pairs, each electrode pair comprising a first and second electrode pair and each electrode pair comprises:
a first molecule bound with the first electrode, the first molecule comprising a first polynucleotide;
a second molecule bound with the second electrode, the second molecule comprising a group configured to intercalate with double stranded polynucleotide compounds; and
wherein, upon contacting the electrode pair with a liquid comprising a second polynucleotide sequence at least partially complementary to the first polynucleotide sequence, the first and second polynucleotide sequences form a duplex region and the intercalating portion intercalates with the duplex region thereby modifying an electrical characteristic of the first and second electrodes whereby the presence of the at least partially complementary second polynucleotide may be determined.

148. The sensor of claim 145, wherein respective, different first polynucleotides are found with the first electrodes of respective, different electrode pairs, whereby the first polynucleotides bound to different first electrodes will selectively form duplex regions with different second polynucleotides.

149. The sensor of claim 145, wherein a distance between the first and second electrodes is less than 500 Angstroms.
